(19) United States
(12) Patent Application Publication
(10) Pub. No.: US 2006/0074108 A1
(43) Pub. Date: Apr. 6, 2006

(54) Matrix metalloprotease (MMP) inhibitors and their application in cosmetic and pharmaceutical composition

(75) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: Bioderm Research, Scottsdale, AZ (US)

(21) Appl. No.: 10/711,775

(22) Filed: Oct. 4, 2004

(51) Int. Cl. A61K 31/444; A61K 31/4184; A61K 31/4172; A61K 31/401; A61K 31/15; A61K 31/12

(52) U.S. Cl. 514/332; 514/355; 514/394; 514/400; 514/639; 514/590; 514/686; 514/680; 514/423; 514/640

(57) ABSTRACT

This invention relates to compounds that are selective inhibitors of Matrix Metalloprotease (also known as Matrix Metalloproteinase, MMP), to cosmetic and pharmaceutical compositions containing them, and to their use in the prevention and/or treatment of ailments associated with MMP, including inflammation, wound healing, skin aging, skin tone discoloration, body odor, oral cavity odor, rosacea, acne, and hair growth modulation.

The present invention relates to compounds that are selective inhibitors of matrix metalloproteases (also known as Matrix Metalloproteinases, MMPs), to cosmetic and pharmaceutical compositions containing them, and to their use in the prevention and/or treatment of ailments associated with MMPs, including inflammation, wound healing, skin aging, skin tone discoloration, body odor, oral cavity odor, rosacea, acne, and hair growth modulation.

Matrix metalloproteases are naturally-occurring enzymes found in most mammals and are zinc-dependent endopeptidases that perform extracellular tissue reorganization (matrix reorganization).

One major biological function of the matrix metalloprotease (MMP) is to catalyze the breakdown of connective tissue or extracellular matrix by virtue of their ability to hydrolyze various components of the tissue or matrix. Examples of the components that may be hydrolyzed by an MMP include collagens (for example, Collagenases type I, II, III, or IV), gelatins (for example, Gelatinases), proteoglycans, and fibronectins. Apart from their role in degrading connective tissue, MMPs are also involved in the activation of the zymogen (pro) forms of other MMPs thereby inducing MMP activation (proenzyme activation). They are also involved in the biosynthesis of TNF-alpha which is implicated in many pathological conditions and can cause or contribute to the effects of inflammation, rheumatoid arthritis, asthma, COPD, autoimmune disease, multiple sclerosis, graft rejection, fibrotic disease, cancer, infectious diseases, malaria, mycobacterial infection, meningitis, fever, psoriasis, cardiovascular/pulmonary effects (e.g., post-ischemic reperfusion injury), congestive heart failure, hemorrhage, coagulation, hyperoxic alveolar injury, radiation damage, cachexia, anorexia, and acute phase responses like those seen with infections and sepsis and during shock (e.g., septic shock and hemodynamic shock).

The “Matrix Metalloproteases” or “MMPs” to which this invention is applicable include all full length mammalian proteases, or a truncated from thereof, or a catalytic domain thereof, that contain a functional metal cation in their active catalytic site. The invention is also applicable to all variants, analogs, orthologs, homologs, and derivatives of such proteases provided they retain their ability to hydrolyze polypeptides and their functional metal cation in their catalytic active site. Recent reviews of MMPs are presented by Albrecht Messerschmidt, Wolfram Bode, and Mirek Cygler (Editors), (2004) Handbook of Metalloproteins, Volume 3, John Wiley, NY; Ivano Bertini, Astrid Sigel, and Helmut Sigel (Editors), (2001) Handbook on Metalloproteins, Marcel Dekker, NY; Woessner and Nagase, (2000) “Matrix metalloproteases and TIMPs”, Oxford University Press, Oxford; and Doherty et al. (2002) Expert Opinion Therapeutic Patents 12(5): 665-707.

Over 30 MMPs have been characterized so far in humans and several major groups have been determined based on substrate specificity, some of which are described below, and are believed applicable to the present invention.

MMP-1(also known as collagenase 1, or fibroblast collagenase). The substrates of MMP-1 include collagen I, collagen II, collagen III, gelatin, and proteoglycans. Over-expression of this enzyme is believed to be associated with emphysema, with hyperkeratosis and atherosclerosis, overexpressed alone in papillary carcinoma.

MMP-2 (also known as gelatinase A, basement membrane collagenase, or proteoglycanase). The substrates of MMP-2 include collagen I, collagen II, collagen IV, collagen V, collagen VII, collagen X, collagen XI, collagen XIV, elastin, fibronectin, gelatin, nidogen, believed to be associated with tumor progression through specificity for type IV collagen (high expression observed in solid tumors and believed to be associated with their ability to grow, invade, develop new blood vessels and metastasize) and to be involved in acute lung inflammation and in respiratory distress syndrome.

MMP-3 (also known as stromelysin 1). The substrates of MMP-3 include collagen III, collagen IV, collagen V, collagen IX, collagen X, laminin, nidogen, overexpression believed to be involved in atherosclerosis, aneurysm and restenosis.

MMP-7 (also known as matrilysin). The substrates of MMP-7 include collagen IV, elastin, fibronectin, gelatin, laminin.

MMP-8 (also known as collagenase 2, or neutrophil collagenase). The substrates of MMP-8 include collagen I, collagen II, collagen III, collagen V, collagen VII, collagen IX, gelatin over-expression of which can lead to non-healing chronic ulcers.

MMP-9 (also known as gelatinase B, or 92 kDa gelatinase). The substrates of MMP-9 include collagen I, collagen III, collagen IV, collagen V, collagen VII, collagen X, collagen XIV, elastin, fibronectin, gelatin, nidogen The above enzyme is believed to be associated with tumor progression through specificity for type IV collagen, to be released by eosinophils in response to exogenous factors such as air pollutants, allergens and viruses, to be involved in the inflammatory response in asthma and to be involved in acute lung inflammation and respiratory distress syndrome. The applicants believe that an inhibitor for this enzyme would be effective in the treatment of chronic obstructive pulmonary disorder (COPD) and/or asthma.

MMP-10 (also known as stromelysin 2). The substrates of MMP-10 include collagen III, collagen IV, collagen V, elastin, fibronectin, and gelatin.

MMP-11 (also known as stromelysin 3). The substrates of MM_{—}11 include serine protease inhibitors (Serpins).

MMP-12 (also known as metalloelastase, human macrophage elastase, or HME). The substrates of MMP-12 include fibronectin, laminin, believed to play a role in tumor growth inhibition and regulation of inflammation and to play a pathological role in emphysema and in atherosclerosis, aneurysm and restenosis. The applicants believe that an inhibitor for this enzyme would be effective in the treatment of chronic obstructive pulmonary disorder (COPD) and/or asthma.

MMP-13 (also known as collagenase 3). The substrates of MMP-13 include collagen I, collagen II, collagen III, collagen IV, collagen IX, collagen X, collagen XIV, fibronectin, and gelatin, recently identified as being overexpressed alone in breast carcinoma. The applicants believe that an inhibitor for this enzyme would be effective in the treatment of breast cancer and arthritis.

MMP-14 (also known as membrane MMP or MT1-MMP). The substrates of MMP-14 include MMP-2, collagen I, collagen II, collagen III, fibronectin, gelatin, laminin.

MMP-15 (also known as MT2-MMP). The substrates of MMP-15 include MMP-2, collagen I, collagen II, collagen III, fibronectin, laminin nidogen.

MMP-16 (also known as MT3-MMP). The substrates of MMP-16 include MMP-2, collagen I, collagen III, fibronectin.

MMP-17 (also known as MT4-MMP), substrates fibrin (fibrinogen).

MMP-18 (also known as collagenase 4).

MMP-19 (also known as Rasi-1). The substrates of MMP-19 include MMP-9, gelatin, laminin-1, collagen IV, and fibronectin.

MMP-20 (also known as enamelysin), substrate amelogenin.

MMP-23 (also known as femalysin), substrate gelatin.

MMP-24 (also known as MT5-MMP). The substrates of MMP-24 include MMP-2, gelatin, fibronectin, chondroitin, and dermitin sulfate proteoglycans.

MMP-25 (also known as MT6-MMP). The substrates of MMP-25 include MMP-2, gelatin, collagen IV, and fibronectin.

MMP-26 (also known as matrilysin 2 or endometase). The substrates of MMP-26 include denatured collagen, fibrinogen, fibronectin, vitronectin.

MMP-28; also known as epilysin, substrates caesin.

Over-activation of a matrix metalloprotease (“MMP”), or an imbalance between an MMP and a natural (i.e., endogenous) tissue inhibitor of a matrix metalloprotease (“TIMP”), has been linked to the pathogenesis of diseases characterized by the breakdown of connective tissue or extracellular matrix. Examples of diseases characterized by over-expression and/or over-activation of an MMP include rheumatoid arthritis, asthma, COPD, osteoarthritis; osteoporosis; periodontitis; multiple sclerosis; gingivitis; corneal, epidermal, and gastric ulceration; atherosclerosis; neointimal proliferation, which leads to restenosis and ischemic heart failure; stroke; renal disease; macular degeneration; and tumor metastasis.

Further, some MMP-mediated diseases may involve over activity of only one MMP enzyme. This is supported by the recent discovery that MMP-13 alone is over-expressed in breast carcinoma, while MMP-1 alone is over-expressed in papillary carcinoma.

“MMP-associated disorder” which is treatable according to the present invention encompasses all disorders in which the expression and/or activity of at least one MMP needs to be decreased irrespective of the cause of such disorders. Such disorders include, for example, those caused by inappropriate ECM degradation. Illustrative but not limiting examples of such MMP-associated disorders are:
Cancer;
Inflammatory disorders such as inflammatory bowel diseases, multiple sclerosis, glomerulonephritis, and uveorentinitis;
Lung diseases such as chronic obstructive pulmonary disorder, asthma, acute lung injury, and acute respiratory distress syndrome;
Dental diseases such as periodontal disease and gingivitis;
Joint and bone diseases such as osteoarthritis and rheumatoid arthritis;
Liver diseases such as liver fibrosis, cirrhosis and chronic liver disease;
Fibrotic diseases such as pulmonary fibrosis, lupus, glomerulosclerosis, systemic sclerosis and cystic fibrosis;
Vascular pathologies such as aortic aneurysm, atherosclerosis, hypertension, cardiomyopathy and myocardial infarction;
Restenosis;
Ophthalmologic disorders such as diabetic retinopathy, dry eye syndrome, macula degeneration and corneal ulceration;
Wound healing disorders such as non healing ulcers, excessive scar formation;
Tissue ulceration such as gastric ulcers and skin ulcers;
Skin disorders such as psoriasis, acne, rosacea, skin discoloration, and skin aging;
Uterus and pregnancy-related disorders such as adenomyosis and pre-eclampsia;
Disorders caused by pathogens such as HIV-1 infection, bacterial meningitis
Central nervous system disorders such as Alzheimer's disease;
Neuroinflammatory disorders such as multiple sclerosis and acute neuroinflammation; and also.
Marfan syndrome, invertebral disk degeneration, graft-versus-host disease and lupus.

Research has been carried out into the identification of inhibitors that are selective, for example, for a few of the MMP subtypes. A MMP inhibitor of improved selectivity would avoid potential side effects associated with inhibition of MMPs that are not involved in the pathogenesis of the disease being treated. Further, use of more selective MMP inhibitors would require administration of a lower amount of the inhibitor for treatment of disease than would otherwise be required and, after administration, partitioned in vivo among multiple MMPs. Still further, the administration of a lower amount of compound would improve the margin of safety between the dose of the inhibitor required for therapeutic activity and the dose of the inhibitor at which toxicity is observed.

The design and therapeutic application of matrix metalloprotease inhibitors was reviewed by Whittaker et al., Chem. Rev., 1999, 99, 2735-2776. The authors explained that the requirement for a molecule to be an effective inhibitor of the MMP class of enzymes is a functional group (e.g. carboxylic acid, hydroxamic acid or sulfhydryl) capable of chelating to the active site zinc II ion, at least one functional group that provides a hydrogen bond interaction with the enzyme backbone, and one or more side chains which undergo effective van der Waals interactions with the enzyme sub sites. A large number of such compounds are mentioned in which chelation is by a hydroxamate group.

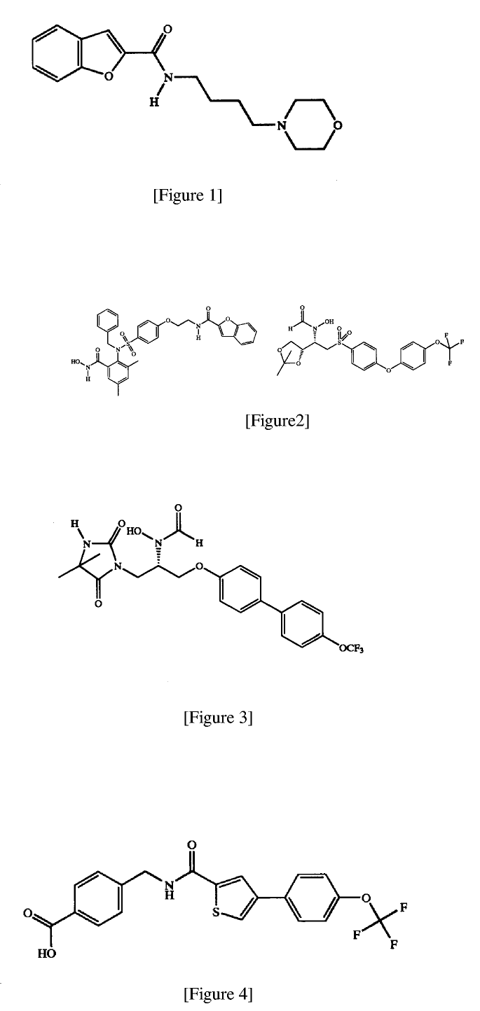

Chen et al., J. Am. Chem. Soc, 2000, 122, 9648-9654 disclose a potent and selective inhibitor for MMP-13. The authors had found that a compound referenced CL-82198 [FIG. 1] exhibited weak inhibition of MMP-13 but complete lack of activity against MMP-1 and MMP-9. Chen at al. postulated that the above compound sits in and extends along the S1′ pocket of MMP-13, with the morpholine group forming a hydrogen bond with the backbone amide group of Leu-82 and with the benzofuran group packing deep into the S1′ pocket, but not binding to zinc of the catalytic domain. The authors decided that the way forward in the design of an MMP-13-selective lead compound was to make a compound that had both a moiety that chelates to zinc of the catalytic domain and a moiety that sits in the S1′ pocket, and arrived at a potent compound called WAY-170523 that shows >5800-, 56- and 500-fold selectivity against MMP-1 and MMP-9.

[FIG. 1]

Stallings et al (WO 01/05389) disclose certain N-hydroxy compounds located adjacent to an aryl ring [FIG. 2]. These compounds have shown strong binding with the catalytic zinc atom in the active-site of MMP.

[FIG. 2]

Further compounds that exhibit selectivity for MMP-12 are described in WO 01/83431 and WO 01/83461 (Shionogi) and are stated to be effective against emphysema and COPD. They rely for activity on the presence of groups that chelate to zinc.

Curtin et al., [Bioorg. Med. Chem. Lett. 11 (2001), 1557-1560] disclose MMP inhibitors bearing a zinc-binding group [FIG. 3], which were reported to be highly selective for MMP-2 versus MMP-1.

[FIG. 3]

Wada et al, [J. Med. Chem., 45, (20020, 219-232], discovered a compound that is selective for the inhibition of MMP-2 and MMP-9 over MMP-1, and which demonstrated antitumor activity in a murine syngenetic tumor growth model. These authors attribute selectivity in MMPs to differences in the depth of the S1′ pocket and classify the MMPs into those with relatively deep pockets (MMP-2, -3, -8, -9, and -13) and those with shallow pockets (MMP-1 and -7). Selectivity is achieved by incorporation of an extended so-called P1′ group such as biphenyl for fitting into the S140 pocket whereas the incorporation of smaller P1′ groups generally leads to broad-spectrum inhibition. Again, the above compounds achieve activity by the presence of groups that chelate to zinc.

Dublanchet et al. (U.S. Patent Application 20040171543) disclose MMP inhibitors based on certain hydroxamic acid derivatives [FIG. 4].

[FIG. 4]

Jarrousse et al. (U.S. Pat. No. 6,645,477) disclose certain MMP and TIMP inhibitors useful for hair growth modulation (i.e. to stimulate hair growth or to retard hair growth).

Wang et al. (U.S. Patent Application 20020037827) disclose the identification of MMP-25 in skin cells and its role in hair growth. The methods for inhibiting MMP-25 activity, leading to the methods useful for inhibiting hair growth are also disclosed.

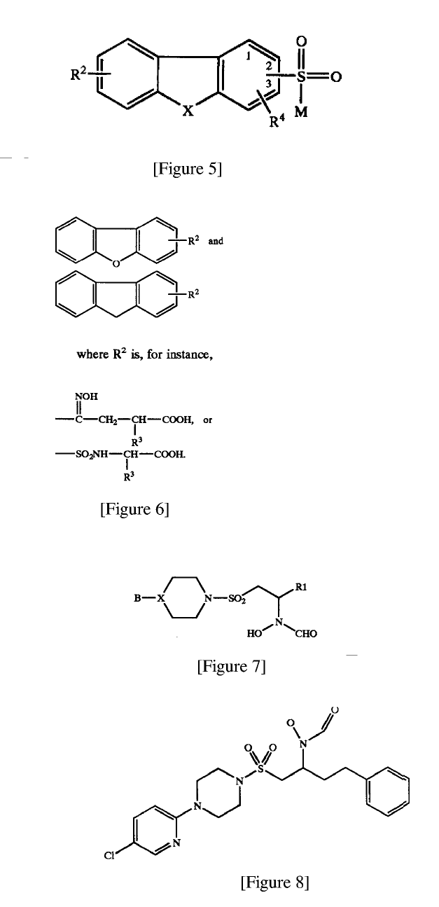

O'Brien et al. (U.S. Patent Application 20040029945) disclose a method of inhibiting MMP using compounds that are dibenzofuran sulfonamide derivatives having the formula in [FIG. 5]. More particularly, O'Brien invention relates to a method of treating diseases in which matrix MMP are involved such as multiple sclerosis, atherosclerotic plaque rupture, restenosis, aortic aneurism, heart failure, periodontal disease, corneal ulceration, burns, decubital ulcers, chronic ulcers or wounds, cancer metastasis, tumor angiogenesis, arthritis, or other autoimmune or inflammatory diseases dependent upon tissue invasion by leukocytes.

[FIG. 5]

Tsuji et al. (U.S. Patent Application 20040175349) report a method of inhibiting hair growth, which comprises administering an inhibitor of elastase-like enzymes or a neutral endopeptidase inhibitor, and use of an inhibitor of elastase-like enzymes or a neutral endopeptidase inhibitor for the preparation of a hair-growth inhibitor.

Newton et al. (U.S. Patent Application 20040176393) provide a method of treating and preventing heart failure and other vascular diseases in a mammal comprising administering an effective amount of a matrix metalloproteinase inhibitor together with a statin. The invention also provides a method for treating and preventing ventricular dilatation comprising administering an effective amount of a MMP inhibitor together with a statin. The MMP inhibitor to be utilized is a substituted bicyclic compound of the formula in [FIG. 6].

[FIG. 6]

Baarlam et al. (U.S. Patent Applications 20040176386 and 20040171641) disclose compounds of the formula in [FIG. 7 and FIG. 8] useful as metalloproteinase inhibitors, especially as inhibitors of MMP 13.

[FIG. 7]

[FIG. 8]

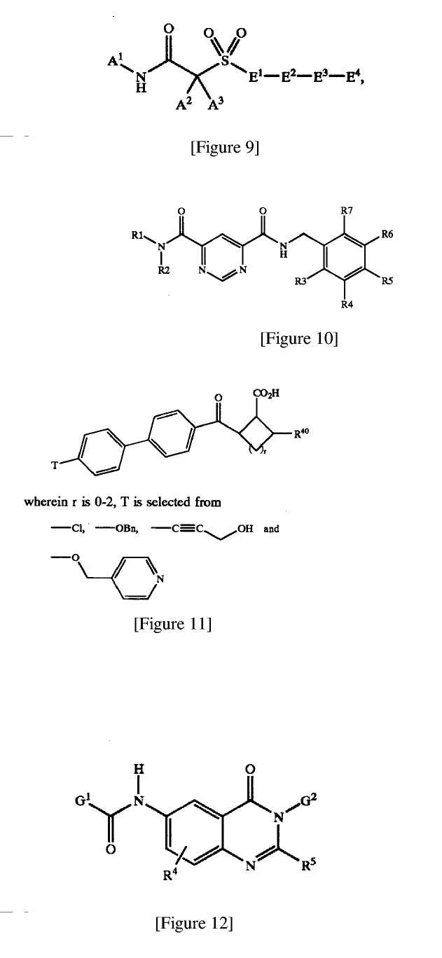

Becker et al. (U.S. Patent Application 20040167182) disclose certain hydroxamic acid and amide compounds (including salts of such compounds), and, more particularly, to aryl- and heteroaryl-arylsulfonylmethyl hydroxamic acids and amides that inhibit protease activity, particularly MMP activity and/or aggrecanase activity. These compounds generally correspond in to structure in [FIG. 9].

[FIG. 9]

Klingler et al. (U.S. Patent Application 20040167120) disclose pyrimidine-4,6-dicarboxylic acid diamides of the formula in [FIG. 10] are suitable for selectively inhibiting collagenase (MMP 13). The pyrimidine-4,6-dicarboxylic acid diamides can therefore be used for treating degenerative joint diseases.

[FIG. 10]

VanZandt et al. (U.S. Patent Application 20040127500) disclose MMP inhibitor compounds that have the generalized formulas as in [FIG. 11].

[FIG. 11]

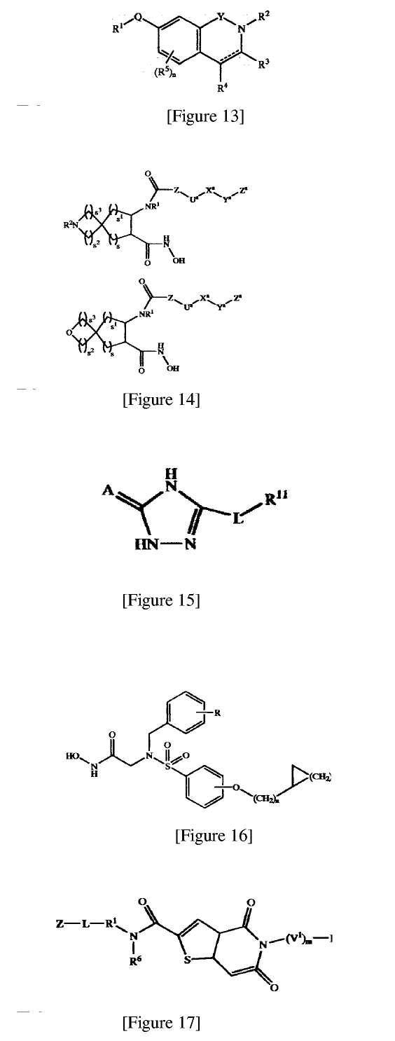

Bunker et al. (U.S. Patent Application 20040142950 and 20040044000) discloses compounds in [FIG. 12 and FIG. 13] that are inhibitors of MMP-13. The compounds are useful for treating diseases mediated by MMP-13, including the diseases recited herein such as breast cancer, cartilage damage, rheumatoid arthritis, and osteoarthritis.

[FIG. 12]

[FIG. 13]

Ott et al. (U.S. Patent Application 20040132693) disclose spiro-cyclic .beta.-amino acid derivatives of formula in [FIG. 14], which are useful as MMP, TNF-.alpha. converting enzyme (TACE), and/or aggrecanase inhibitors.

[FIG. 14]

King et al. (U.S. Patent Application 20040116491) disclose hydantoin derivatives of formula in [FIG. 15], which are useful as inhibitors of MMP, TNF-.alpha. converting enzyme (TACE), aggrecanase, or a combination thereof.

[FIG. 15]

Monroe et al. (U.S. Patent Application 20040105897) disclose composition containing one or more of zinc ions, calcium ions, rubidium ions and/or potassium ions in a pharmaceutically acceptable carrier, which, when administered to a patient in need thereof, effectively modulates the activity of at least MMP-2 and/or MMP-9 in the wound. These inventors have identified MMP-2 and MMP-9 in increased quantities in certain medical conditions. In one such medical condition, MMPs have been noted to be involved both in the peripheral region and particularly within the deep recesses of a chronic wound. It has also been a noted increase in these MMPs in “difficult to heal” open wounds. Further the present inventors have discovered a synthesized composition which, when clinically introduced to a site exhibiting the presence of one or more MMPs effectively shuts down the activity of MMPs. This therapeutic effect is particularly evident with respect to the modulation of MMP-2 and MMP-9, as evidenced by analysis of wound cultures for the presence of MMPs 2 and 9, and resulting visually observable improvement in the healing of the wound.

Hayakawa et al (U.S. Patent Application 20040082630) certain .alpha.-amino-N-hydroxy-acetamide derivatives of formula in [FIG. 16], wherein R is di-lower alkyl amino, 1,2,3-triazol-2yl or 1,2,4-triazol-4-yl, m represents an integer from 1 up to and including 10, and n represents an integer from 0 up to and including 10, and the use of such hydroxamic acid derivatives as medicaments, and a method of treating conditions or diseases mediated by MMPs using said derivatives.

[FIG. 16]

Johnson et al. (U.S. Patent Application 20040063673) disclose pharmaceutical compositions comprising a compound of formula in [FIG. 17], together with a pharmaceutically acceptable carrier that provide methods of inhibiting an MMP-13 enzyme.

[FIG. 17]

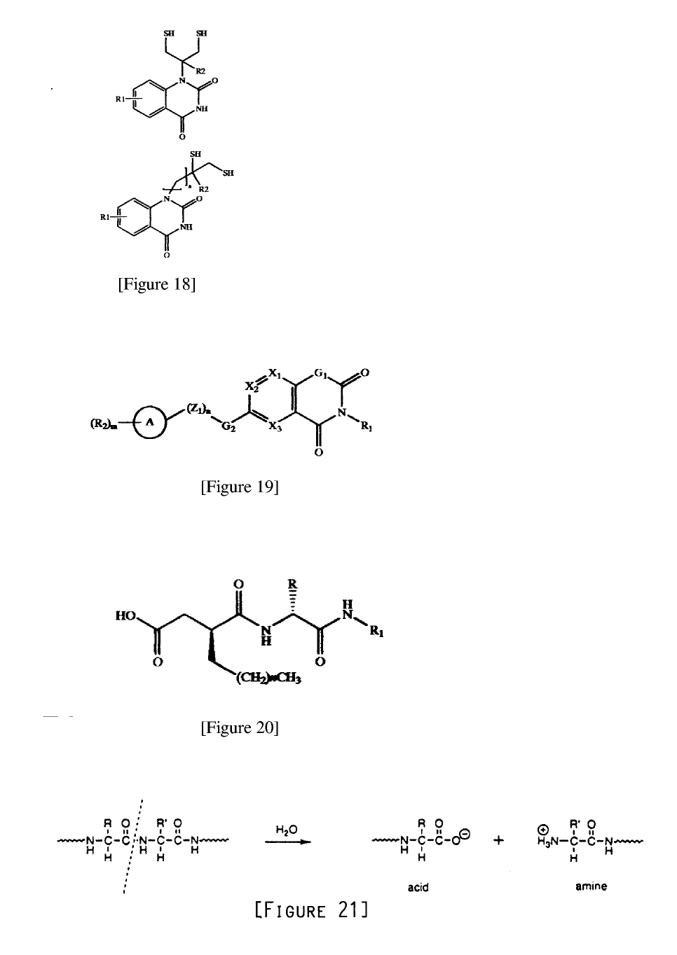

Heinicke et al. (U.S. Patent Application 20040044013 and 20040023953) disclose certain dimercaptoalkyl-substituted quinazoline-2,4(1H,3H)diones [FIG. 18]. Compounds of this substance class show a surprisingly clear and thus pharmacologically interesting MMP-inhibitory effect.

[FIG. 18]

Gaudilliere et al. (U.S. Patent Application 20040006077) disclose certain thiazine and oxazine derivatives [FIG. 19] as MMP-13 inhibitors.

[FIG. 19]

Arnold et al. (U.S. Patent Application 20030225272) disclose certain N-[2(R)-Nonylsuccinic acid]-L-tyrosine-N-2-(N-morpholino)ethylamide; N-[2(R)-Nonylsuccinic acid]-L-phenylalanine-N-3-(N-morpholino)propylamide-N-[2(R)-Nonylsuccinic acid]-L-valine-N-2-(N-morpholino)ethylamide; N-[2(R)-Nonylsuccinic acid]-L-tyrosine-N-(4-methoxyphenyl)amide; N-[2(R)-Nonylsuccinic acid]-L-phenylalanine-N-(4-methoxyphenyl)amide; N-[2(R)-Nonylsuccinic acid]-L-norvaline-N-(4-methoxyphenyl)amide; N-[2(R)-Nonylsuccinic acid]-L-arginine-N-(4-methoxyphenyl)amide; N-[2(R)-Nonylsuccinic acid]-L-phenylglycine-N-methylamide; N-[2(R)-Nonylsuccinic acid]-L-tyrosine-N-cyclopentylamide; and N-[2(R)-Nonylsuccinic acid]-L-tyrosine-N-3-dimethylaminopropylamide [FIG. 20] useful as MMP-2 and MMP-9 inhibitors.

[FIG. 20]

Among other recent prior art disclosures, Li (U.S. Patent Application 200400439830 and 20040038960), Picard (U.S. Patent Application 200400439790), Ortwine (U.S. Paten Application 200400389740), Nahra et al. (U.S. Patent Application 200400389730), Bunker et al. (U.S. Patent Application 20040038961 and 20040038959), Roark (U.S. Patent Application 20040034009), Frescos et al. (U.S. Patent Application 20040024024), and Getman et al. (U.S. Patent Application 20040034071) teach additional methods for inhibiting various MMP for the control of ailments associated with MMPs.

Varani et al. (U.S. Patent Application 20040034098) disclose that chronological aging of human skin can be delayed with the topical application of an MMP inhibitor, preferably a retinoid (an indirect MMP inhibitor). Retinoids also normalize procollagen biosynthesis. Chronological aging, or natural aging, is evidenced in elderly (80+ years old) skin by increased MMP levels and decreased procollagen levels when compared with younger individuals. Prophylactic treatment of not yet chronologically-aged skin with a retinoid both inhibits degradation of dermal collagen and restores procollagen synthesis.

Quirk (U.S. Patent Applications 20040127420 and 20030166567) report inhibitors of MMP useful for treating wounds. The inhibitors are peptides having sequences related to cleavage regions of the proenzyme forms of MMP. The peptide inhibitors of the invention can be formulated into therapeutic compositions and wound dressings that facilitate healing.

Additional references for prior art methods for MMP inhibition and their applications in medicine include: Agren, M. S. (1999). Matrix metalloproteases (MMPs) are required for re-epithelialization of cutaneous wounds. Arch. Dermatol. Res. 291, 583-590; Becker, J. W., Marcy, A. I., Rokosz, L. L., Axel, M. G., Burbaum, J. J., Fitzgerald, P. M., Cameron, P. M., Esser, C. K., Hagmann, W. K., Hermes, J. D., and Springer, J. P. (1995). Stromelysin-1: Three dimensional structure of the inhibited catalytic domain and of the C-truncated proenzyme. Protein Sci. 4, 1966-76; Brown, R L., Breeden, M P., and Greenhalgh, M D., (1994). PDGF and TGF-a act synergistically to improve wound healing in the genetically diabetic mouse. J. Surg. Res. 56, 562-570; Browner, M. F., Smith, W. W., Castelhano, A. L. (1995). Matrilysin-inhibitor complexes: Common themes among 18 metalloproteinases. Biochemistry 34, 6602-10; Di Colandrea, T., Wang, L., Wille, J., D'Armiento, J., and Chada, K. K. (1998). Epidermal expression of collagenase delays wound healing in transgenic mice. J. Invest. Dermatol. 111, 1029-1033; Duivenvoorden, W. C. M., Hirte, H. W., and Singh, G. (1997). Use of tetracycline as an inhibitor of matrix metalloproteinase activity secreted by human bone metastasizing cancer cells. Invasion and Metas. 17, 312-322; Fernandez-Catalan, C., Bode, W., Huber, R., Turk, D., Calvete, J. J., Lichte, A., Tschesche, H., and Maskos, K. (1998). Crystal structure of the complex formed by membrane type-I matrix metalloproteinase with the tissue inhibitor of metalloproteinases-2, the soluble progelatinase A receptor. EMBO J. 17, 5238-48; Freire, E., van Osdol, W W., Mayorga, O L, and Sanchez-Ruiz, J M. (1990). Calorimetrically determined dynamics of complex unfolding transitions in proteins. Annu Rev Biophys Biophys Chem. 19, 159-88; Gomis-Ruth, F. X., Maskos, K., Betz, M., Bergner, A., Huber, R., Suzuki, K., Yoshida, N., Nagase, H., Brew, K., Bourenkov, G. P., Bartunik, H., and Bode, W. (1997). Mechanism of inhibition of the human matrix metalloproteinase stromelysin-1 by TIMP-1. Nature 389, 77-81; Grams, F., Reinemer, P., Powers, J. C., Kleine, T., Pieper, M., Tschesche, H., Huber, R., Bode, W. (1995). X-ray structures of human neutrophil collagenase complexed with peptide hydroxamate and peptide thiol inhibitors: Implications for substrate binding and rational drug design. Eur. J. Biochem. 228, 830-834; Guex, N. and Peitsch, M. C. (1997). Swiss Model and the Swiss-PdbViewer: An environment for comparative protein modeling. Electrophoresis 18, 2714-2723; Higgins, D G., Bleasby, A J., and Fuchs, R. (1992). CLUSTAL V: improved software for multiple sequence alignment. Comput Appl Biosci., 8(2), 189-91; Howard, E. W., Bullen, E. C., and Banda, M. J. (1991). Preferential inhibition of 72 and 92 kDa gelatinase by tissue inhibitor of metalloproteinase-2. J. Biol. Chem. 266, 13070-13075; Huang, W., Suzuki, K., Nagase, H., Arumugam, S., Van Doren, S. R., and Brew, K. (1996). Folding and characterization of the amino terminal domain of human tissue inhibitor of metalloproteinases-1 (TIMP-1) expressed at high yield in E. coli. FEBS Lett. 384, 155-161; Karlsson, R., and Falt, A. (1997). Experimental design for kinetic analysis of protein-protein interactions with surface plasmon resonance biosensors. J. Immunol. Meths. 200,121-33; Lakowicz, J. R. (1983). Principles of Fluorescence Spectroscopy, Chapter 10, Plenum Press, New York, London; Levit, S., and Berger, A. (1976). Ribonuclease S-peptide. A model for molecular recognition. J. Biol. Chem. 251, 1333-9; Levy, D. E., Lapierre, F., Liang, W., Ye, W., Lange, C. W., Li, X., Grobelny, D., Casabonne, M., Tyrrell, D., Holme, K., Nadzan, A., and Galardy, R. E. (1998). Matrix metalloproteinase inhibitors: A structure activity study. J. Med. Chem. 41, 199-223; Li, J., Brick, P., O'Hare, M. C., Skarzynski, T., Lloyd, L. F., Curry, V. A., Clark, I. M., Bigg, H. F., Hazleman, B. L., Cawston, T. E., et al.(1995). Structure of full-length porcine synovial collagenase reveals a C-terminal domain containing a calcium-linked, four-bladed beta-propeller. Structure 3, pp. 541-49; Libson, A. M., Gittis, A. G., Collier, I. E., Marmer, B. L., Goldberg, G. I., and Lattman, E. E. (1995). Crystal structure of the haemopexin-like C terminal domain of gelatinase A. Nat. Struct. Biol. 2, 938-42; Lofas, S., Johnsson, B., Tegendahl, K., and Ronnberg, I. (1993). Dextran modified gold surfaces for surface plasmon resonance biosensors; immunoreactivity of immobilized antibodies and antibody-surface interaction studies. J. Colloid Interface Sci. 65, 423-431; Morton, T. A., Myska, D. G., and Chaiken, I. M. (1995). Interpreting complex binding kinetics from optical biosensors: A comparison of analysis by linearization, the integrated rate equation, and numerical integration. Anal. Biochem. 227, 176-185; Moses, M. A., Marikovsky, M., Harper, J. W., Vogt, P., Eriksson, E., Klagsbrun, M. and Langer, R. (1996). Temporal study of the activity of matrix metalloproteinases and their endogenous inhibitors during wound healing. J. Cell. Biochem. 60, 379-386; Odake, S., Morita, Y., and Morikawa, T. (1994). Inhibition of matrix metalloproteinases by peptidyl hydroxamic acids. Biochem. Biophys. Res. Comm. 199, 1442-1446; Olson, M. W., Gervasi, D. C., Mobashery, S., and Fridman, R. (1997). Kinetic analysis of the binding of human matrix metalloproteinase 2 and 9 to tissue inhibitor of metalloproteinase (TIMP)-1 and TIMP-2. J. Biol. Chem. 272, 29975-29983; O'Shannessy, D. J., Brigham-Burke, M., Soneson, K. K, Hensley, P., and Brooks, I. (1993). Determination of rate and equilibrium binding constants for macromolecular interactions using surface plasmon resonance: use of non linear least squares analysis methods. Anal. Biochem. 212, 457-468; Reinemer, P., Grams, F., Huber, R., Kleine, T., Schnierer, S., Pieper, M., Tschesche, H., Bode, W. (1994). Structural implications for the role of the N terminus in the superactivation of collagenases: A crystallographic study. FEBBS Lett. 338, 227-33; Saarialho-Kere, U. K. (1998). Patterns of matrix metalloproteinase and TIMP expression in chronic ulcers. Arch. Dermatol. Res. 290 (suppl), 47-54; Sayle, R. A. and Milner-White, E. J. (1995). RasMol: Biomolecular graphics for all. Trends in Biochemical Sciences 20, 374-376; Segel, I H. (1993) Enzyme Kinetics: Behavior and analysis of rapid equilibrium and steady-state enzyme systems. Wiley Classics Library, John Wiley and Sons, Inc. New York; Su, J-L., Becherer, D., Edwards, C., Bukhart, W., McMgeehan, G. M., and Champion, B. R. (1995). Monoclonal antibodies against human collagenase and stromelysin. Hybridoma. 14, 383-390; Taylor, K. B., Windsor, J. L., Caterina, N. C. M., Bodden, M. K., and Engler, J. A. (1996). The mechanism of inhibition of collagenase by TIMP-1. J. Biol. Chem. 271, 23938-23945; Tuuttila, A., Morgunov, E., Bergmann, U., Lindqvist, Y., Maskos, K., Fernandez-Catalan, C., Bode, W., Tryggvason, K., and Schneider, G. (1998). Three dimensional structure of human tissue inhibitor of metalloproteinases-2 at 2.1.ANG. resolution. J. Mol. Biol. 284, 1133-1140; Vaalamo, M., Weckroth, M., Puolakkainen, P., Kere, J., Saarinen, P., Lauharanta, J., and Saarialho-Kere, U. K. (1996). Patterns of matrix metalloproteinase and TIMP-1 expression in chronic and normally healing human cutaneous wounds. Brit. J. Dermatol. 135, 52-59; Vaalamo, M., Mattila, L., Johansson, N., Kariniemi, A-L., Karjalainen-Lindsberg, -L., Kahari, V-M., and Saarialho-Kere, U. K. (1997). Distinct populations of stromal cells express collagenase-3 (MMP-13) and collagenase-1 (MMP-1) in chronic ulcers, but not in normally healing wounds. J. Investig. Dermatol. 109, 96-101; Weckroth, M., Vaheri, A., Lauharanta, J., Sorsa, T., and Konttinen, Y. T. (1996). Matrix metalloproteinases, gelatinases, and collagenases in chronic leg ulcers. J. Investig. Dermatol. 108, 1119-1124; Wojtowicz-Praga, S. M., Dickson, R. B., and Hawkins, M. J. (1997). Matrix metalloproteinase inhibitors. Investigational new Drugs. 15, 61-75. These references are included to show the great amount of prior art effort in this area, which has still not provided a satisfactory solution to this problem.

There is a need for further inhibitors of MMPs that exhibit selectivity for individual enzymes or for groups of enzymes, as this could be utilized to develop novel cosmetic and pharmaceutical compositions containing them, and to their use in the prevention and/or treatment of ailments associated with MMPs, including inflammation, wound healing, skin aging, skin tone discoloration, body odor, rosacea, acne, and hair growth modulation.

### DETAILED DESCRIPTION

Proteases catalyze amide (peptide) bond hydrolysis in protein or peptide substrates [FIG. 20].

[FIG. 20]

Proteases are classified by (a) their site of action, such as exopeptidases and endopeptidases, or (b) by their reaction mechanisms and nature of active-site residues involved in such mechanisms, such as serine proteases, cysteine proteases, aspartyl proteases, and zinc proteases (also called metalloproteases). The serine and cysteine proteases act directly as nucleophiles to attack the substrate. The aspartyl and zinc proteases activate water molecules as the direct attacking species on the peptide bond. For example, in case of zinc proteases (zinc MMP) one atom of Zn++ is coordinated to two histidine and one glutamic acid side chains in the active-site. On water molecule binds with activated Zn site to form Zn—OH, which is then ready to attack the substrate peptide bond. Once the substrate protein is bound to the active site, zinc can coordinate to the carbonyl oxygen of the peptide bond to be attacked, lowering barriers electronically for [HO—] attack. A conserved glutamate side chain in the active site now acts as catalytic base to protonate the amine product as it leaves the site [FIG. 22].

[FIG. 22]

The substrate analogs that have strong coordination sites for zinc can be potent, selective inhibitors of zinc proteases. There is a need for further inhibitors of MMPs that exhibit selectivity for individual enzymes or for groups of enzymes, as this could be utilized to develop novel cosmetic and pharmaceutical compositions containing them, and to their use in the prevention and/or treatment of ailments associated with MMPs, including inflammation, wound healing, skin aging, skin tone discoloration, body odor, rosacea, acne, and hair growth modulation.

We have now discovered certain MMP inhibitors that are selectively binding with zinc and contain novel chelating groups or atomic centers. Thus, we have now discovered that, surprisingly and unexpectedly, an alkyl ketone side chain directly attached to an aromatic ring that also contains a hydroxyl group in a position next to the ketone side chain attachment (i.e. an alpha-hydroxyaryl alkyl ketone), or a nitrogen hetero-aromatic alkyl ketone with the alkyl ketone side chain directly attached to the hetero-aromatic ring at a position alpha to the nitrogen heteroatom, or one of the nitrogen atoms of hetero-aromatic rings if such rings contain more than one nitrogen in the hetero-aromatic ring, provide chelating centers required for selective binding with zinc active-site of various MMPs. Moreover, certain derivatives, such as oximes and hydrazides, of such hydroxyaryl alkyl ketones and nitrogen hetero-aromatic alkyl ketones also possess selective chelating or binding properties with the zinc active-site of MMPs. The novel MMP inhibitors of the present invention do not appear to act as competitive substrates, but distort the geometry of one of zinc centers in MMPs by binding with such zinc cations in the form of a five or six-member ring with one or two double bonds, respectively, in a bidentate structure form. After distorting the geometry of such zinc cations, the MMP inhibitors of the present invention appear to move away from thus “deactivated” active-site and go to the next active-site to deactivate it. In this manner, the MMP inhibitors of the present invention regenerate and recycle themselves.

Because they coordinate with the functional zinc cation of the MMP, prior art inhibitors are competitive with binding of the endogenous substrate. As the concentration of an enzyme's substrate rises, the potency of a competitive inhibitor for the active site of the enzyme diminishes. This is a disadvantage for a pharmaceutical agent, as a rising concentration of substrate will eventually reduce the agent's therapeutic efficacy. However, the regenerative process of the MMPs of the present invention described above circumvents this limitation of prior art MMP inhibitors.

MMP inhibitors typically mimic the natural substrates in that they coordinate the functional zinc cation and occupy from 1 to 3 specificity binding pockets along the enzyme active site. As there is much structural similarity among these binding pockets of the various MMPs, this binding mode generally requires a greater modulation of site-specificity of their chemical binding sites of the inhibitor molecule to achieve better inhibitor-MMP selectivity.

If MMP inhibitors bind allosterically to an enzyme or group of enzymes, then they should exhibit improved selectively because they do not employ the coordination to zinc that is a common feature amongst MMPs. Furthermore, a noncompetitive or uncompetitive MMP inhibitor could also bind to MMP-TIMP complexes and may not suffer diminishing binding potency in the presence of a rising concentration of substrate. Accordingly, a noncompetitive or uncompetitive MMP inhibitor that binds to an MMP-TIMP complex should maintain its therapeutic efficacy in the presence of a rising substrate concentration. A further advantage of a noncompetitive or uncompetitive MMP inhibitor is that when the inhibitor is bound to an MMP-TIMP complex and the TIMP disassociates from the complex to provide free TIMP and inhibitor-bound MMP, the MMP remains inhibited. Although such allosteric MMP inhibitors have been disclosed in the prior art, for example, Dublanchet et al. (U.S. Patent Application 20040171543) the MMPs of Dublanchet et al. remain coordinated to zinc ions and do not regenerate. Moreover, a non-competitive or uncompetitive MMP inhibitor does not have a strong binding hence can be displaced by other molecules, including substrates for MMPs.

In one aspect, the present invention provides a compound that is a matrix metalloprotease inhibitor, and that (a) binds into at least one or both of the binding sites of MMP to effect the spatial distortion of such active-sites, and (b) exhibits selectivity for a matrix metalloprotease or group of matrix metalloproteases, and (c) detaches itself from the bound state with the zinc center of the active-site of MMP after distorting its spatial configuration, and (d) repeats the cycle for effecting the spatial distortion of the active-site of additional MMP.

A compound meeting the above requirements of the present invention may have a molecular weight in the range 100-850 and comprise 1-4 ring systems, one of which is an aryl with at least one hydroxyl group, and contains a ketone group attached to an alkyl group on one side and the aromatic ring on the other side. The ketone group is attached to aryl moiety at a position alpha to the hydroxyl group on aryl moiety.

A compound meeting the above requirements of the present invention may have a molecular weight in the range 100-850 and comprise 1-4 ring systems, one of which is an aryl with at least one hydroxyl group, and contains a ketone group attached to an alkyl group on one side and the aromatic ring on the other side. The ketone group is attached to aryl moiety at a position alpha to the hydroxyl group on aryl moiety, and the ketone group is further transformed into an oxime or hydrazide derivative.

The compound may also have a molecular weight in the range 100-850 and comprise 1-4 ring systems, one of which is a hetero-aromatic ring with at least one nitrogen atoms in the hetero-aromatic ring, and which also contains a ketone group attached to an alkyl group on one side and the hetero-aromatic ring on the other side. The ketone group is attached to hetero-aromatic moiety at a position alpha to at least one nitrogen atom in hetero-aromatic ring moiety.

The compound may also have a molecular weight in the range 100-850 and comprise 1-4 ring systems, one of which is a hetero-aromatic ring with at least one nitrogen atoms in the hetero-aromatic ring, and which also contains a ketone group attached to an alkyl or substituted alkyl group on one side and the hetero-aromatic ring on the other side. The ketone group is attached to hetero-aromatic moiety at a position alpha to at least one nitrogen atom in hetero-aromatic ring moiety, and the ketone group is further transformed into an oxime or hydrazide derivative.

This invention further provides a method of prevention and/or treatment of ailments caused by or associated with MMPs, which comprises administering to said patient a compound as defined above.

This invention further provides a method of treating or preventing inflammation or inflammatory responses, including allergic responses and allergies.

This invention further provides a method of treating or preventing rheumatoid arthritis or osteoarthritis associated with over-expression of MMP-3 and/or MMP-9.

This invention further provides a method of wound healing.

This invention further provides a method of modulation of hair growth (hair growth promotion or retardation).

This invention further provides a method of treating or preventing acne.

This invention further provides a method of treating or preventing rosacea.

This invention further provides a method of treating or preventing skin aging.

This invention further provides a method of treating or preventing skin tone discoloration.

This invention further provides a method of treating or preventing body odor.

This invention further provides a method of treating or preventing oral cavity odor and gingivitis.

A number of hydroxy acetophenone compositions obtained from natural plant sources have been disclosed in the prior art with antioxidant and other benefits. For example, acetophenone derivatives such as Paeonol (3-hydroxy-5-methoxy acetophenone), 2,5-Dihydroxy-4-Methoxy Acetophenone, and 2,5-Dihydroxy-4-Methyl Acetophenone, have been obtained from Chinese peony. Quinacetophenone (2-acetyl hydroquinone) has been obtained from primrose (Primula Ovalifolia). Scutellarin and Scutellarein (hydroxy benzopyranones) have been obtained from Scutellaria plants. Xanthoxyline (2-hydroxy-4,6-dimethoxyacetophenone) has been isolated from Sebastiania schottiana. Acetophenone derivatives, such as 1-(3-Hydroxy-4-methoxy-5-methylphenyl) ethanone and 1-(3-hydroxy-4-methoxyphenyl) ethanone have been identified from stem bark of Lamprothamnus zanguebaricus. Apocynin (4-hydroxy-3-methoxyacetophenone), is a well-known acetophenone derivative isolated from the traditional medicinal plant Picrorhiza kurroa. 4-Hydroxyacetophenone has been obtained from Ligularia vellerea. These acetophenone derivatives are known for their antioxidant, microcirculation improvement, anti-inflammatory, MAO inhibition, and histamine suppression benefits.

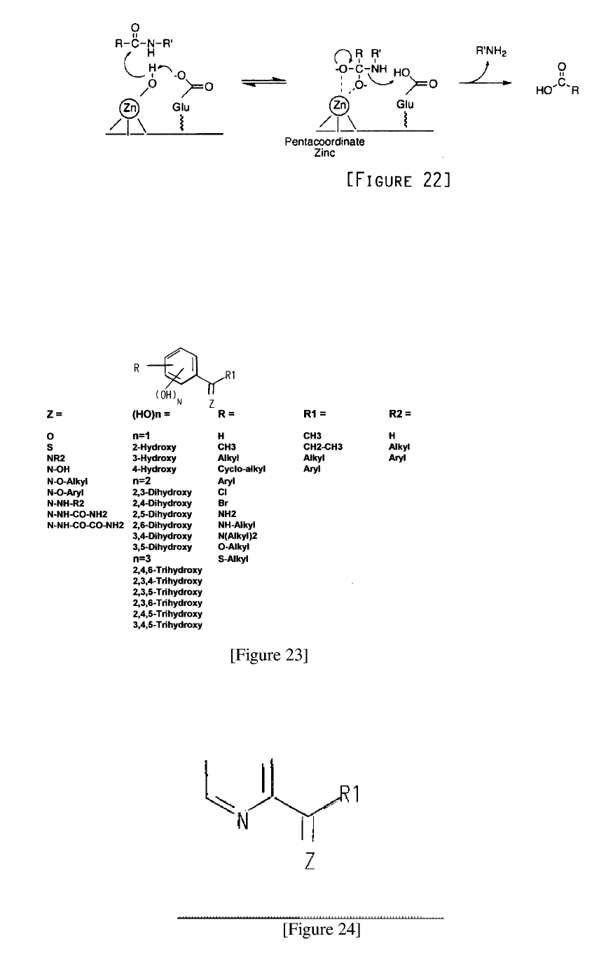

Surprisingly and unexpectedly, it has now been found that these acetophenones and their derivatives and certain hydroxyaryl alkyl ketones, substituted hydroxyaryl alkyl ketones, and their derivatives provide excellent MMP inhibition benefits. Moreover, conversion of carbonyl groups of said acetophenone and hydroxyaryl alkyl ketones into their oxime or hydrazone or samicarbazone or oxamic hydrazone derivative still maintains the MMP inhibitory effect. These chemical structures are included in [FIG. 23]. R, in these examples, also includes alkylamino- and alkylaminoalkyl-substituents and their salt derivatives.

[FIG. 23]

This is both surprising and unexpected since oxime derivatives of certain hydroxyaryl alkyl ketones have been disclosed in U.S. Patent Application 20030049287 (Ley at al.) as antioxidants, and not as MMP inhibitors.

The hydroxyaryl alkyl ketones of the present invention can have additional cyclic rings attached at the aromatic moiety. Such attached rings can be alicyclic, aromatic, heterocyclic, or a combination thereof in nature. Examples include 1-hydroxy-2-acetylnaphthalene; 1-hydroxy-2-acetyl-5,6,7,8-tetrahydro-naphthalene; 7-acetyl-8-hydroxyquinoline; 3-acetyl-4-hydroxyacridine; 6-acetyl-7-hydroxybenzothiazole. As can be appreciated by any one versed in the art that a very large number of compounds that have the structural criteria discovered in the present invention is possible, and not all such structures can be included herein.

We have additionally discovered that the alkyl ketone or substituted alkyl ketone moiety can also be attached to a nitrogen hereto-aromatic ring at a position adjacent to the ring nitrogen atom. Such compounds also show selective MMP inhibitory effect; as such compounds can also bind with zinc cation of the active-site and cause distortion of the spatial configuration of the active-site. Such spatial distortions cause an inhibitory effect for MMP activity. The five- and six-member hetero-aromatic ring of the acyl- or alkyl ketone-substituted MMP inhibitors of the present invention can have additional hetero-atoms in their ring structure. For example, additional nitrogen atoms, or sulfur or oxygen atoms, or a combination thereof, can additionally be present. The examples of hetero-aromatic ring structures include 2-acetylpyridine, 2-acetylpyrrole, 2-acetylimidazole, 2-acetylthiazole, 2-acetylpyrimidine, 2-acetylindole, 2-acetyl-1-methylpyrrole, 2-acetyl-4-methylpyridine, 1-acetylphenothiazine, 2-hydroxy-1-acetylphenothiazine, 8hydroxy-9-acetylphenanthrene, 2-acetylpyrazine, 2-acetylquinoline, 2-acetyl-8-hydroxyquinoline, 2-acetyltryptophane, 2-acetyltryptophanamide, 2-acetylpyridine N-oxide, 2-acetylquinazoline, 2-acetylquinoxaline, 3-acetylpyridazine, 6,6′-diacetyl-2,2′-pyridyl, 3-actyl-1,2,4-trizol, and their other acetyl side chain substituted and/or hetero-aromatic ring substituted derivatives. These chemical structure criteria are further illustrated in partial ring structures represented in [FIG. 24]. Moreover, the conversion of the ketone moiety of such hetero-atom ketones into their oxime or hydrazone or samicarbazone or oxamic hydrazone derivative still maintains the MMP inhibitory effect.

[FIG. 24].

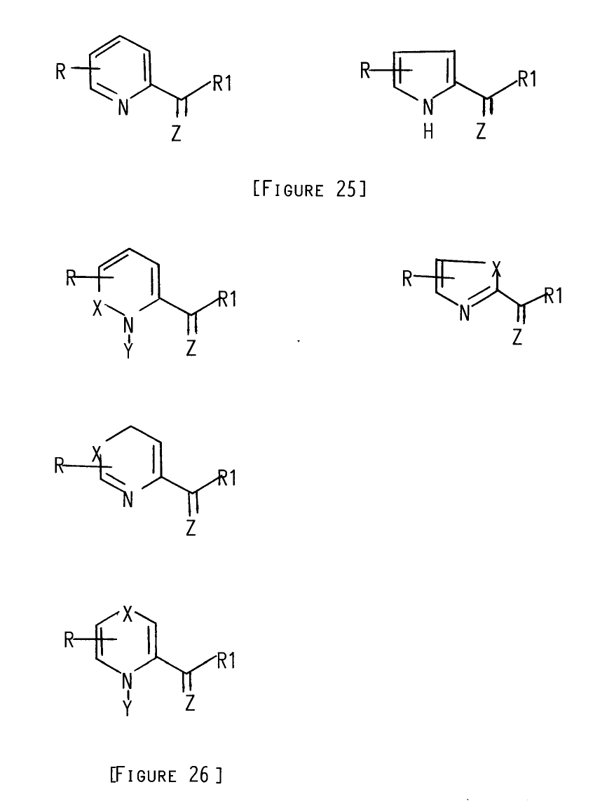

Additional examples of chemical structures of present invention based on five and six-member nitrogen heteroatom alkyl ketones and their derivatives are included in [FIG. 25].

[FIG. 25]

Additional examples of chemical structures of present invention based on additional hetero-atom ring substituted five and six-member nitrogen heteroatom alkyl ketones and their derivatives are included in [FIG. 26].

[FIG. 26]

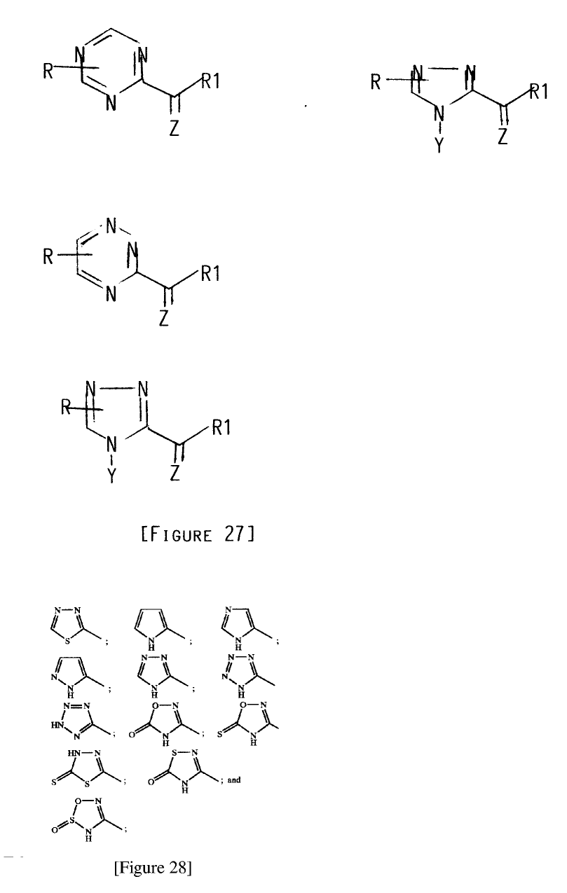

It should be additionally noted that, as should be clear to those versed in the art, additional heteroatomic substituents in the nitrogen heteroatom ring with an alkyl ketone moiety or their derivatives also possess MMP inhibition properties. For example, in case of five- and six-member nitrogen heteroatom rings in which two additional nitrogen heteroatoms are included [FIG. 27] the compounds still maintain their MMP inhibitory effect. A large variation in five- and six-member multi-heteroatom ring structures is thus possible. A number of such examples can be noted from U.S. Patent Applications 20040142950, 20040063673, and 20040116491, among others. Examples of additional five-member ring structures with an alkyl ketone moiety are included in [FIG. 28].

[FIG. 27]

[FIG. 28]

It should be noted that various substituents in [FIG. 24] to [FIG. 27] are as identified in [FIG. 23], with additional annotations; X═S, O, NH, or N—Y; Y═H or alkyl.

The precise mechanism by which the MMP of the present invention operate is not known. In one aspect, the present invention provides a compound that is a matrix metalloprotease inhibitor, and that (a) binds into at least one or both of the Zinc binding sites of MMP to effect the spatial distortion of such active-sites, and (b) exhibits selectivity for a matrix metalloprotease or group of matrix metalloproteases, and (c) detaches itself from the bound state with the zinc center of the active-site of MMP after distorting its spatial configuration, and (d) repeats the cycle for effecting the spatial distortion of the active-site of additional MMP. The spatial distortion of zinc active-site may be caused by the electron donating hydroxyl group of hydroxyaryl moiety and the ketone group of alkyl ketone moiety of a hydroxyaryl alkyl ketone compound, as illustrated in [FIG. 29]. Similar donation of electrons by the nitrogen hetero-atom of the aromatic nitrogen heterocyclic moiety and ketone group of alkyl ketone moiety of an N-hetero-aromatic alkyl ketone compound can cause similar distortions of the zinc active-sites of MMP. In any event, these results are both surprising and unexpected, irrespective of the actual mechanism of such MMP inhibitory effects elicited by the compounds of the present invention.

[FIG. 29]

Although a number of specific examples of compounds that are useful as MMP inhibitors of the present invention are included herein, a large number of additional such compounds can be obtained from Sigma Aldrich Company, Catalog 2003-2004, by performing a chemicals search based on sub-structures criteria specified herein. This information is also available on the internet at www.sigmaaldrich.com, by performing a chemicals search based on sub-structures criteria specified herein.

Application in Cosmetic and Pharmaceutical Compositions

A wide scope of applications in cosmetic and pharmaceutical compositions has been discovered for the compounds of the present invention. Some of these applications, which include Wound Care; Hair Growth Modulation; Skin Aging; Arthritis; Acne; Topical Inflammation; Body Odor; and Oral Odor, are further illustrated in the Examples section herein.

It should become clearer in the later discussion that some of these applications are interrelated. This does not prevent the utility or decrease the value of the present invention.

Wound Care.

The process by which tissue repair takes place is termed wound healing and is comprised of a continuous sequence of inflammation and repair, in which epithelial, endothelial, inflammatory cells, platelets and fibroblasts briefly come together outside their normal domains, interact to restore a semblance of their usual discipline and having done so resume their normal function.

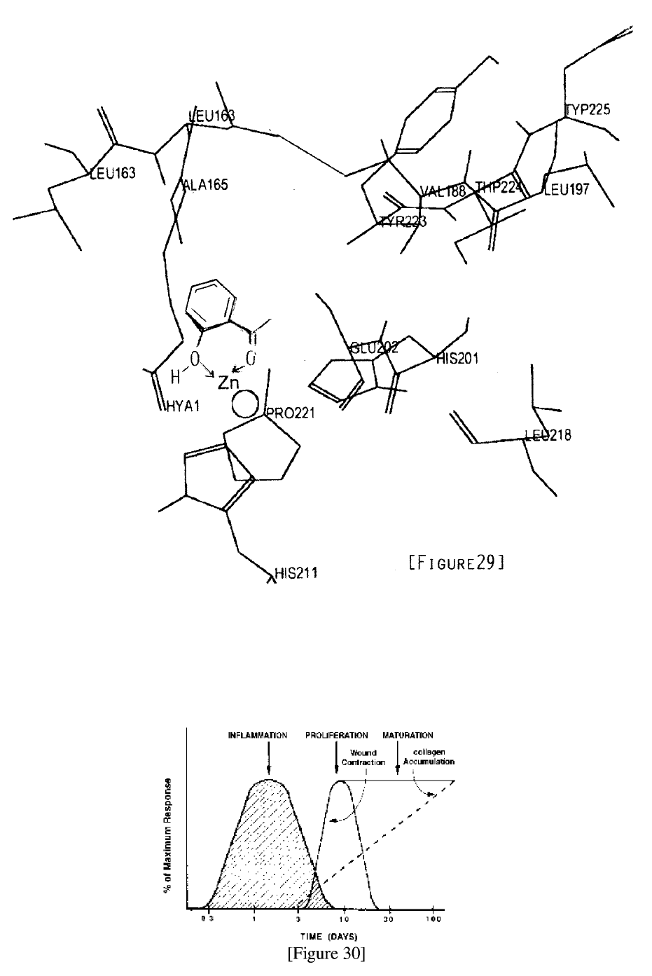

The process of wound repair differs little from one kind of tissue to another and is generally independent of the form of injury. Although the different elements of the wound healing process occur in a continuous, integrated manner, it is convenient to divide the overall process into three overlapping phases and several natural components for descriptive purposes [FIG. 30].

[FIG. 30]

Inflammatory Phase (Day 0-5).

The healing response is initiated at the moment of injury. Surgical or traumatic wounds disrupt the tissue architecture and cause hemorrhage. Initially, blood fills the wound defect and exposure of this blood to collagen in the wound leads to platelet degranulation and activation of Hageman factor. This in turn sets into motion a number of biological amplification systems including the complement kinin and clotting cascades and plasmin generation. These serve to amplify the original injury signal and lead not only to clot formation, which unites the wound edges, but also to the accumulation of a number of mitogens and chemoattractants at the site of wounding.

Production of both kinins and prostaglandins leads to vasodilatation and increased small vessel permeability in the region of the wound. This results in oedema in the area of the injury and is responsible for the pain and swelling which occurs early after injury. Within 6 h, circulating immune cells start to appear in the wound. Polymorphonuclear leucocytes (PMN) are the first blood leucocytes to enter the wound site. They initially appear in the wound shortly after injury and subsequently their numbers increase steadily, peaking at 24-48 h. Their main function appears to be phagocytosis of the bacteria which have been introduced into the wound during injury. The presence of PMN in the wound following injury does not appear to be essential in order for normal wound healing to take place, with healing proceeding normally in their absence provided that bacterial contamination has not occurred. In the absence of infection, PMN have a relatively short life span in the wound and their numbers decrease rapidly after the third day.

The next cellular, immune element to enter the wound are macrophages. These cells are derived from circulating monocytes by a combination of migration and chemotaxis. They first appear within 48-96 h post-injury and reach a peak around the third day post-injury. These macrophages have a much longer life span than the PMN and persist in the wound until healing is complete. Their appearance is followed somewhat later by T-lymphocytes, which appear in significant numbers around the fifth day post-injury, with peak numbers occurring about the seventh day after injury. In contrast to PMN, the presence and activation of both macrophages and lymphocytes in the wound is critical to the progress of the normal healing process.

Macrophages just like neutrophils phagocytose and digest pathological organisms and tissue debris. In addition, macrophages release a plethora of biologically active substances. Many of these substances facilitate the recruitment of additional inflammatory cells and aid the macrophage in tissue decontamination and debridement; in addition growth factors and other substances are also released which are necessary for the initiation and propagation of granulation tissue formation. These intercellular transmitters are known collectively as cytokines.

Proliferative Phase (Day 3-14).

In the absence of significant infection or contamination the inflammatory phase is short, and after the wound has been successfully cleared of devitalized and unwanted material it gives way to the proliferative phase of healing. The proliferative phase is characterized by the formation of granulation tissue in the wound. Granulation tissue consists of a combination of cellular elements, including fibroblasts and inflammatory cells, along with new capillaries embedded in a loose extra cellular matrix of collagen, fibronectin and hyaluronic acid. Fibroblasts first appear in significant numbers in the wound on the third day post-injury and achieve peak numbers around the seventh day. This rapid expansion in the fibroblast population at the wound site occurs via a combination of proliferation and migration. Fibroblasts are derived from local mesenchymal cells, particularly those associated with blood vessel adventitia, which are induced to proliferate and attracted into the wound by a combination of cytokines produced initially by platelets and subsequently by macrophages and lymphocytes. Fibroblasts are the primary synthetic element in the repair process and are responsible for production of the majority of structural proteins used during tissue reconstruction. In particular, fibroblasts produce large quantities of collagen, a family of triple-chain glycoproteins, which form the main constituent of the extracellular wound matrix and which are ultimately responsible for imparting tensile strength to the scar. Collagen is first detected in the wound around the third day post-injury, and thereafter the levels increase rapidly for approximately 3 weeks. It then continues to accumulate at a more gradual pace for up to 3 months post wounding. The collagen is initially deposited in a seemingly haphazard fashion and these individual collagen fibrils are subsequently reorganized, by cross-linking, into regularly aligned bundles oriented along the lines of stress in the healing wound. Fibroblasts are also responsible for the production of other matrix constituents including fibronectin, hyaluronic acid and the glycosaminoglycans. The process of fibroblast proliferation and synthetic activity is known as fibroplasia.

Revascularization of the wound proceeds in parallel with fibroplasia. Capillary buds sprout from blood vessels adjacent to the wound and extend into the wound space. On the second day post-injury, endothelial cells from the side of the venule closest to the wound begin to migrate in response to angiogenic stimuli. These capillary sprouts eventually branch at their tips and join to form capillary loops, through which blood begins to flow. New sprouts then extend from these loops to form a capillary plexus. The soluble factors responsible for angiogenesis remain incompletely defined. It appears that angiogenesis occurs by a combination of proliferation and migration. Putative mediators for endothelial cell growth and chemotaxis include cytokines produced by platelets, macrophages and lymphocytes in the wound, low oxygen tension, lactic acid and biogenic amines. Of the potential cytokine mediators of neovascularization basic fibroblast growth factor (bFGF), acidic FGF (aFGF), transforming growth factors-a and b (TGF-a and -b) and epidermal growth factor (EGF) have all been shown to be potent stimuli for new vessel formation. FGF, in particular, has been shown to be a potent inducer of in vivo neovascularization.

While these events are proceeding deep in the wound, restoration of epithelial integrity is taking place at the wound surface. Re-epithelialization of the wound begins within a couple of hours of the injury. Epithelial cells, arising from either the wound margins or residual dermal epithelial appendages within the wound bed, begin to migrate under the scab and over the underlying viable connective tissue. The epidermis immediately adjacent to the wound edge begins thickening within 24 h after injury. Marginal basal cells at the edge of the wound loose their firm attachment to the underlying dermis, enlarge and begin to migrate across the surface of the provisional matrix filling the wound. Fixed basal cells in a zone near the cut edge undergo a series of rapid mitotic divisions, and these cells appear to migrate by moving over one another in a leapfrog fashion until the defect is covered. Once the defect is bridged, the migrating epithelial cells loose their flattened appearance, become more columnar in shape and increase in mitotic activity. Layering of the epithelium is re-established and the surface layer eventually keratinized. Reepithelialization is complete in less than 48 h in the case of approximated incised wounds, but may take substantially longer in the case of larger wounds where there is a significant tissue defect. If only the epithelium is damaged, such as occurs in split thickness skin graft donor sites, then repair consists primarily of re-epithelization with minimal or absent fibroplasia and granulation tissue formation. The stimuli for re-epithelization remain incompletely determined, but it appears that the process is mediated by a combination of loss of contact inhibition, exposure of constituents of the extracellular matrix, particularly fibronectin, and by cytokines produced by immune mononuclear cells. EGF, TGF-b, bFGF, platelet-derived growth factor (PDGF) and insulinlike growth factor-I (IGF-I) in particular, have been shown to promote epithelialization.

Maturation Phase (Day 7 to 1 Year).

Almost as soon as the extracellular matrix is laid down, its reorganization begins. Initially, the extracellular matrix is rich in fibronectin, which forms a provisional fibre network. This serves not only as a substratum for migration and ingrowth of cells, but also as a template for collagen deposition by fibroblasts. There are also significant quantities of hyaluronic acid and large molecular weight proteoglycans present, which contribute to the gel-like consistency of the extracellular matrix and aid cellular infiltration. Collagen rapidly becomes the predominant constituent of the matrix. The initially randomly distributed collagen fibres become cross-linked and aggregated into fibrillar bundles, which gradually provide the healing tissue with increasing stiffness and tensile strength. After a 5-day lag period, which corresponds to early granulation tissue formation and a matrix largely composed of fibronectin and hyaluronic acid, there is a rapid increase in wound breaking strength due to collagen fibrogenesis. The subsequent rate of gain in wound tensile strength is slow, with the wound having gained only 20% of its final strength after 3 weeks. The final strength of the wound remains less than that of uninjured skin, with the maximum breaking strength of the scar reaching only 70% of that of the intact skin.

This gradual gain in tensile strength is due not only to continuing collagen deposition, but also to collagen remodelling, with formation of larger collagen bundles and alteration of intermolecular crosslinking. Collagen remodelling during scar formation is dependent on both continued collagen synthesis and collagen catabolism. The degradation of wound collagen is controlled by a variety of collagenase enzymes, and the net increase in wound collagen is determined by the balance of these opposing mechanisms. The high rate of collagen synthesis within the wound returns to normal tissue levels by 6-12 months, while active remodelling of the scar continues for up to 1 year after injury and indeed appears to continue at a very slow rate for life.

As remodelling progresses, there is a gradual reduction in the cellularity and vascularity of the reparative tissue which results in the formation of a relatively avascular and acellular collagen scar. Grossly this can be observed as a reduction in erythema associated with the earlier scar and some reduction in the scar volume, resulting in a pale thin scar. This is normally a desirable feature of healing; however, in some cases shrinkage of the scar may give rise to an undesirable reduction in skin mobility resulting in contracture.

Wound contraction, i.e. inward movement of the wound edge, is a further important element in the healing process and should be distinguished from contracture. Sharply incised wounds without significant tissue loss, approximated early after injury, heal rapidly without the need for significant reduction in the wound volume. Such wounds are described as having healed by primary intention. Large wounds, however, particularly those associated with significant tissue loss, heal by secondary intention, with granulation tissue gradually filling the defect and epithelization proceeding slowly from the wound edges. Contraction of the wound edges can lead to a significant reduction in the quantity of granulation tissue required to fill the wound defect and a reduction in the area requiring reepithelization, with a consequent reduction in scar volume. Contraction is only undesirable where it leads to unacceptable tissue distortion and an unsatisfactory cosmetic result. Although contraction normally accounts for a larger part of overall wound closure in looseskinned animals, it still accounts for a significant proportion of the healing process in man, particularly in areas where the skin is not tightly bound down to underlying structures, such as on the back, neck and forearms. Initially following injury, where the wound edges are not approximated, there is a slight retraction of the wound edges due to the release of normal elastic tension in the skin, with a resultant increase in wound volume. The wound area starts to decrease rapidly from the third day onwards. While this is due in part to reepithelization, the main reason is an inward movement of the uninjured skin edges. Wound contraction usually begins around the fifth day postwounding and is complete by 12-15 days after wounding. Fibroblasts within the wound appear to be responsible for providing the force for this contractile activity. It was initially felt that specialized fibroblasts called myofibroblasts provided the motive force for wound contraction via a musclelike cell contraction. More recent studies reveal that wound contraction occurs as a result of an interaction between fibroblast locomotion and collagen reorganization. The contraction is thought to be mediated via the attachment of collagen fibrils to cell surface receptors, with the resulting tractional forces generated by cell motility bringing the attached collagen fibrils closer together and eventually compacting them.

The regulation of wound contraction remains poorly defined. Information regarding the effects of specific cytokines on contraction is limited and often conflicting. TGF-b has been found to promote contraction even in the absence of serum; PDGF has also been found to either increase contraction or have no effect, while both FGF and EGF have been found by different authors to either have no effect or cause a moderate enhancement of contraction.

Scar Formation

As mentioned previously, the process of wound healing is essentially similar in all tissues and is relatively independent of the mode of injury; however, slight variation in the relative contribution of the different elements to the overall result may occur. The final product of the healing process is a scar. This relatively avascular and acellular mass of collagen serves to restore tissue continuity, strength and function. Delays in the healing process cause the prolonged presence of wounds, while abnormalities of the healing process may lead to abnormal scar formation. Successful completion of wound healing may not always yield the desired clinical result, particularly where the final cosmetic appearance of the scar is of primary importance.

From this discussion it is clear that wound healing process is quite complex, and it requires a timed management of inflammation, reduction of MMP action to stop destruction of freshly synthesized proteinaceous tissue, and collagen and elastin synthesis. A combination of the compounds of the present invention can achieve anti-inflammatory effect, MMP inhibition, and collagen and elastin synthesis enhancement. Such formulations are described in the Examples section of this invention.

Hair Growth Modulation (Hair Growth Promotion or Hair Growth Retardation).

In humans, the growth and renewal of the hair are mainly determined by the activity of the hair follicles and by their dermo-epidermal environment. Their activity is cyclic and essentially comprises three phases, i.e. the anagenic phase, the catagenic phase and the telogenic phase.

The active anagenic phase or growth phase, which lasts for several years and during which the hair gets longer, is followed by a very short and transient catagenic phase that lasts a few weeks, and then comes a rest phase, known as the telogenic phase, which lasts a few months.

At the end of the rest period, the hair falls out and another cycle begins. The head of hair is thus under constant renewal, and out of the approximately 150,000 hairs which make up a head of hair, at any given moment, approximately 10% of them are at rest and will thus be replaced within a few months.

In a large number of cases, early hair loss occurs in individuals who are genetically predisposed, and it usually affects men. This more particularly concerns androgenetic or androgenic or even androgenogenetic alopecia.

This alopecia is essentially due to a disruption in hair renewal which leads, in a first stage, to an acceleration of the frequency of the cycles, at the expense of the quality of the hair and then at the expense of its quantity. A gradual depletion of the head of hair takes place by regression of the so-called “terminal” hairs at the downy stage. Regions are preferentially affected, in particular the temples or frontal bulbs and the back of the head in men, whilst in women diffuse alopecia of the vertex is observed.

Substances for suppressing or reducing alopecia, and in particular for inducing or stimulating hair growth or reducing hair loss, have been sought for many years in the cosmetics and pharmaceutical industries.

Admittedly, in this respect, a large number of very diverse active compounds have already been proposed, such as, for example, 2,4-diamino-6-piperidinopyrimidine 3-oxide or “Minoxidil” described in U.S. Pat. No. 6,645,477 (Jarrousse et al.) and U.S. Pat. No. 4,596,812 (Chidsey et al.), or the many derivatives thereof, such as those described, for example, in patent applications EP 0 353 123, EP 0 356 271, EP 0 408 442, EP 0 522 964, EP 0 420 707, EP 0 459 890 and EP 0 519 819. It has thus been discovered that a metalloprotease inhibitor, or any functional biological equivalent, makes it possible to induce and/or stimulate the growth of head hair or other hairs, and/or to reduce their loss in an effective manner. For example, Jarrousse et al. disclosed that metalloproteases are present in the internal structures of hair follicles, namely in the inner epithelial sheath (IRS). In particular, MMP-9 is found in the IRS.

Metalloproteases (MMPs) are members of a family of proteolitic enzymes (endoproteases) which contain a zinc atom coordinated to 3 cysteine residues and one methionine residue in their active site and which degrade the macromolecular components of the extracellular matrix and the basal sheets at neutral pH (collagen, elastin, etc.). These enzymes, which are very widely distributed in the living world, are present, but weakly expressed, in normal physiological situations such as organ growth and tissue renewal. However, their overexpression in man and their activation are associated with many processes which involve the destruction and remodelling of the matrix. This entails, for example, an uncontrolled resorption of the extracellular matrix.

Metalloproteases are produced and secreted in an inactive zymogenic form (pro-enzyme). These zymogenic forms are then activated in the extracellular environment by the removal of a propeptide region. The members of this family can activate each other.

Regulation of the activity of MMPs thus takes place at the level of the expression of the genes (transcription and translation), at the level of the activation of the zymogenic form, or at the level of the local control of the active forms.

The main regulators of the activity of MMPs are the tissue inhibitors of metalloproteases, or TIMPs. However, the expression of MMPs is also modulated by growth factors, cytokines, oncogenic products, or matrix constituents.

Now, it is known that in the course of the hair cycle, hair follicles pass from a low-level location in the dermis in the anagenic phase, to a high-level location in the dermis during the telogenic phase. This movement should be accompanied by a change in the extracellular matrix which allows the migration of the follicle, this change possibly being due to an expression of the MMPs, bringing about a controlled degradation of the said extracellular matrix. It is at the end of the telogenic phase that hair loss occurs. However, it is also known that cytokines and growth factors have an influence on the hair cycle. For example, epidermal growth factor (EGF) promotes the in vitro transition from the anagenic phase to the catagenic phase (formation of a “club” structure characteristic of the catagenic phase), this being the phase which precedes the loss of the head hairs or other hairs. It is also known that there is an inflammatory phase in alopecia. MMPs, and particularly MMP-9 can be induced by interleukin-1 and/or EGF, in particular in the fibroblasts of the dermal papillae. The advantage of reducing the expression of MMPs in the scalp in order to slow down or inhibit the degradation of the perifollicular matrix and thus to slow down or even prevent hair loss may thus be appreciated.

The compounds of the present invention also relate to the use, in or for the preparation of a composition, of an effective amount of at least one metalloprotease inhibitor or of any functional biological equivalent, which is intended to induce and/or stimulate the growth of head hair or other hairs and/or to slow down their loss.

Inflammation.

The major causes of physical disability (arthritis, osteoporosis, stroke, lupus, inflammatory bowel disease, asthma, allergy), mental deterioration (Alzheimer's disease, Vascular dementia, depression, Parkinson's disease), and death (cardiovascular disease, diabetes, cancer), all are initiated and propagated by systemic inflammation. Under normal conditions inflammation is a response to injury and has a major role in immune function and tissue repair. A dysregulation of the inflammatory mechanism may occur with aging or infection, and the influence of environmental and genetic factors. Mediators of inflammation such as C-reactive protein, cytokines, adhesion molecules, and metaloptoteinases may also contribute to the development and progression of inflammatory processes. Thus reduction of levels of inflammatory markers may indicate amelioration of the inflammatory process and reduced risk for inflammatory diseases. A number of antiinflammatory drugs are currently used and new agents are being developed for the prevention and treatment of inflammatory disorders. Antiinflammatory agents are the most widely used class of medications world-wide. The major drugs with antiinflammatory action are nonsteroidal antiinflammatory drugs (NSAIDS), steroids, acetaminophen (COX-3 inhibitors), 5-lipoxygenase inhibitors, leukotriene receptor antagonists, leukotriene A4 hydrolase inhibitors, angiotensin converting enzyme antagonists, beta blockers, antihistaminics, histamine 2 receptor antagonists, phosphodiesterase-4 antagonists, cytokine antagonists, CD44 antagonists, antineoplastic agents, 3-hydroxy-3-methylglutaryl coenzyme A inhibitors (statins), estrogens, androgens, antiplatelet agents, antidepressants, Helicobacter pylori inhibitors, proton pump inhibitors, thiazolidinediones, dual-action compounds, combinations of these drugs with other agents, derivatives and metabolites of synthetic and natural antiinflammatory agents. These are further disclosed by Thomas, U.S. Patent Application 20040176469.

The role of MMP inhibitors in the control of inflammation has already been discussed herein. We have now discovered, surprisingly, that the MMP inhibitors of the present invention can also block cyclooxygenase enzymes, COX-1, COX-2, and COX-3 for the prevention of inflammation. The enzyme cyclooxygenase (COX) catalyzes the first step of the synthesis of prostanoids. As reported by Hinz et al., “Cyclooxygenase-2, 10-years later”, Pharmacology and Experimental Therapeutics, volume 300, issue 2, 367-375 (2002), in the early 1990s COX was demonstrated to exist as two distinct isoforms. COX-1 is constitutively expressed as a “housekeeping” enzyme in most tissues. By contrast, COX-2 can be up-regulated by various pro-inflammatory agents, including lipopolysaccharide, cytokines, and growth factors. Whereas many of the side effects of nonsteroidal anti-inflammatory drugs (NSAIDs) (e.g., gastrointestinal ulceration and bleeding, platelet dysfunctions) are caused by a suppression of COX-1 activity, inhibition of COX-2-derived prostanoids facilitates the anti-inflammatory, analgesic, and antipyretic effects of NSAIDs. During the past few years specific inhibitors of the COX-2 enzyme have emerged as important pharmacological tools for treatment of pain and arthritis. The COX isoenzymes share a 60% identity in their amino acid sequence. The structure of the COX proteins consists of three distinct domains: an N-terminal epidermal growth factor domain, a membrane-binding motif, and a C-terminal catalytic domain that contains the COX and peroxidase active sites. The COX active site lies at the end of a hydrophobic channel that runs from the membrane-binding surface of the enzyme into the interior of the molecule. NSAIDs act at the COX active site in several ways. Aspirin irreversibly inactivates both COX-1 and COX-2 by acetylating an active-site serine, this covalent modification interferes with the binding of arachidonic acid at the COX active site. By contrast, reversible competitive inhibitors of both isoforms (e.g., mefenamate, ibuprofen) compete with arachidonic acid for the COX active site. A third class of NSAIDs (e.g., flurbiprofen, indomethacin) causes a slow, time-dependent reversible inhibition of COX-1 and COX-2, which results from the formation of a salt bridge between the carboxylate of the drug and arginine 120 followed by conformational changes. It has now been discovered that the MMP inhibitor of the present invention, in addition to their anti-inflammatory effect by their MMP inhibition, also cause an anti-inflammatory effect by the binding of their ketone group in their alkyl ketone substituents with arginine at amino acid position 120 to form a Schiff's base, which results in the spatial distortion of the active site of COX enzymes resulting in their inactivation. This is both surprising and unexpected that the hydroxyaryl- and nitrogen hetero-aromatic alkyl ketones of the present invention provide such a dual benefit as anti-inflammatory agents by their both MMP inhibition and COX inhibition.

MMP inhibitors of the present invention can be formulated in various cosmetic and pharmaceutical consumer products utilizing a variety of delivery systems and carrier bases. Such consumer product forms include the group consisting of shampoos, after shaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

In another preferred aspect, the delivery system or a carrier base are selected in the form of a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, impregnated or coated diaper, and similar delivery or packaging form.

In another preferred aspect, the delivery system can be human body or hair deodorizing solution, deodorizing powder, deodorizing gel, deodorizing spray, deodorizing stick, deodorizing roll-on, deodorizing paste, deodorizing cream, deodorizing lotion, deodorizing aerosol, and other commonly marketed human body and hair deodorizing compositions, household deodorizing solution, deodorizing powder, deodorizing gel, deodorizing spray, carpet deodorizer, room deodorizer, and other commonly marketed household deodorizing compositions, animals and pets deodorizing solution, deodorizing powder, deodorizing gel, deodorizing spray, animals and pets carpet deodorizer, animals and pets room deodorizer, and other commonly marketed animal and pet deodorizing compositions.

In another preferred aspect, the delivery system can be traditional water and oil emulsions, suspensions, colloids, microemulsions, clear solutions, suspensions of nanoparticles, emulsions of nanoparticles, or anhydrous compositions.

Additional cosmetically or pharmaceutically beneficial ingredients can also be included in the formulated compositions of the present invention, which can be selected from, but not limited to skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

In another preferred aspect, the cosmetically acceptable composition further comprises one or more excipient selected from the group consisting of water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C.sub.10 to C.sub.22, long chain fatty amines from about C.sub.10 to C.sub.22, fatty alcohols, ethoxylated fatty alcohols and di-tail phospholipids.

Representative saccharides include nonionic or cationic saccharides such as agarose, amylopectins, amyloses, arabinans, arabinogalactans, arabinoxylens, carageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitin, chitins, chitosan, chitosan pyrrolidone carboxylate, chitosan glycolate chitosan lactate, cocodimonium hydroxypropyl oxyethyl cellulose, colominic acid ([poly-N acetyl-neuraminic acid]), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomainans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragancanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, steardimonium hydroxyethyl cellulose, raffinose, rhamsan, tapioca starch, whelan, levan, scleroglucan, sodium alginate, stachylose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994) which is incorporated entirely by reference. Complex carbohydrates found in Kirk-Othmer Encyclopedia of Chemical Technology, Fourth Edition, Vol. 4, John Wiley and Sons, NY pp. 930-948, 1995 which is herein incorporated by reference.

The cosmetically acceptable composition of this invention may include surface-active agents. Surface active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents, and dispersion polymers.

Anionic surface-active agents useful herein include those disclosed in U.S. Pat. No. 5,573,709, incorporated herein by reference. Examples include alkyl and alkyl ether sulfates. Specific examples of alkyl ether sulfates which may be used In this invention are sodium and ammonium salts of lauryl sulfate, lauryl ether sulfate, coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide.

Another suitable class of anionic surface-active agents is the alkyl sulfuric acid salts. Important examples are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, for example, sulfur trioxide or oleum, obtained according to known sulfonation methods, including bleaching and hydrolysis. Preferred are alkali metal and ammonium sulfated C.sub.12-38 n-paraffins.

Additional synthetic anionic surface-active agents include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Preferred anionic surface-active agents for use in the cosmetically acceptable composition of this invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecyl benzene sulfonate. [Para 175]Amphoteric surface-active agents which may be used in the cosmetically acceptable composition of this invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOL. as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Quaternary ammonium compounds can also be used in the cosmetically acceptable composition of this invention as long as they are compatible in the compositions of the invention, wherein the structure is provided in the CTFA Cosmetic Ingredient Handbook, Fifth Edition, 1988, page 40. Cationic surface-active agents generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof.

Examples of quaternary ammonium compounds include but are not limited to: Behentrimonium chloride, Cocotrimonium chloride, Cethethyldimonium bromide, Dibehenyidimonium chloride, Dihydrogenated tallow benzylmonium chloride, disoyadimonium chloride, Ditallowdimonium chloride, Hydroxycetyl hydroxyethyl dimonium chloride, Hydroxyethyl Behenamidopropyl dimonium chloride, Hydroxyethyl Cetyidimonium chloride, Hydroxyethyl tallowdimonium chloride, myristalkonium chloride, PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryidimonium chloride, cetalkonium chloride, dicetyidimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The cosmetically acceptable compositions may include long chain fatty amines from about C.sub.10 to C.sub.22 and their derivatives. Specific examples include dipalmitylamine, lauramidopropyldimethylamine, and stearamidopropyl dimethylamine. The cosmetically acceptable compositions of this invention may also include fatty alcohols (typically monohydric alcohols), ethoxylated fatty alcohols, and di-tail phospholipids, which can be used to stabilize emulsion or dispersion forms of the cosmetically acceptable compositions. They also provide a cosmetically acceptable viscosity. Selection of the fatty alcohol is not critical, although those alcohols characterized as having fatty chains of C.sub.10 to C.sub.32, preferably C.sub.14 to C.sub.22, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof. Phospholipids such as phosphatidylserine and phosphatidylcholine, and mixtures thereof may also be included. When used, the fatty alcohol component is included in the formulations at a concentration of about 1 to about 10 weight percent, more preferably about 2 to about 7 weight percent.

Nonionic surface-active agents, which can be used in the cosmetically acceptable composition of the present invention, include those broadly defined as compounds produced by the condensation of alkylene oxide groups (hydrophilic in nature) with an organic hydrophobic compound, which may be aliphatic or alkyl aromatic in nature. Examples of preferred classes of nonionic surface-active agents are: the long chain alkanolamides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surface-active agents such as betaines can also be useful in the cosmetically acceptable composition of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH.sub.2).sub.3 radical is attached to the nitrogen atom of the betaine are also useful in this invention.

The anionic, cationic, nonionic, amphoteric or zwitterionic surface-active agents used in the cosmetically acceptable composition of this invention are typically used in an amount from about 0.1 to 50 percent by weight, preferably from about 0.5 to about 40 percent by weight, more preferably from about 1 to about 20 percent by weight.

The cosmetically acceptable composition of this invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant is present in the composition at concentrations of from about 0.5 to about 40 percent by weight, preferably from about 0.5 to about 20 percent by weight and more preferably from about 0.5 to about 12 percent by weight.

The cosmetically acceptable composition of this invention may include petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, vaseline solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Corning, Midland, Mich., USA. Additional alkyl and phenyl silicones may be employed to enhance moisturizing properties. Resins such as dimethicone (and) trimethylsiloxysilicate or Cyclomethicone (and) Trimethylsiloxysilicate fluid, may be utilized to enhance film formation of skin care products. When used, the petrolatum, wax or hydrocarbon or oil component is included in the formulations at a concentration of about 1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetically acceptable composition of this invention may include fatty ester emollients, which are listed in the International Cosmetic Ingredient Dictionary, Eighth Edition, 2000, p. 1768 to 1773. Specific examples of suitable fatty esters for use in the formulation of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C.sub. 12 to C.sub. 16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, and caprylic/capric triglycerides. When used the fatty ester emollient is preferably included in the formulations of this invention at a concentration of about 1 to about 8 weight percent, more preferably about 2 to about 5 weight percent.

The compositions of this invention may also include silicone compounds. Preferably, the viscosity of the silicone component is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane endblocked with trimethyl units, is one preferred example. Dimethicone having a viscosity between 50 and 1,000 cps is particularly preferred. When used, the silicone oils are preferably included in the formulations of this invention at a concentration of 0.1 to 5 weight percent, more preferably 1 to 2 weight percent.

The cosmetically acceptable compositions of this invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane. 200 fluid having a viscosity of about 1 centistoke. When used, the silicone oils are preferably included in the formulations of this invention at a concentration of 0.1 to 30 weight percent, more preferably 1 to 20 weight percent.

The cosmetically acceptable compositions of this invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., and from General Electric, Waterford, N.Y., USA. When used, the silicone oils are preferably included in the formulations of this invention at a concentration of 0.1 to 30 weight percent, more preferably 1 to 20 weight percent.

Silicone surfactants or emulsifiers with polyoxyethylene or polyoxypropylene side chains may also be used in compositions of the current invention. Preferred examples include dimethicone copolyols and 5225C Formulation Aids, available from Dow Corning, Midland, Mich., USA and Silicone SF-1528, available from General Electric, Waterford, N.Y., USA. The side chains may also include alkyl groups such as lauryl or cetyl. Preferred are lauryl methicone copolyol. 5200 Formulation Aid, and cetyl dimethicone copolyol, known as Abil EM-90, available from Goldschmidt Chemical Corporation, Hopewell, Va. Also preferred is lauryl dimethicone, known as Belsil LDM 3107 VP, available from Wacker-Chemie, Munchen, Germany. When used, the silicone surfactants are preferably included in the formulations of this invention at a concentration of 0.1 to 30 weight percent, more preferably 1 to 15 weight percent. Amine functional silicones and emulsions may be utilized in the present invention. Preferred examples include Dow Corning 8220, Dow Corning 939, Dow Corning 949, Dow Corning 2-8194, all available from Dow Corning, Midland, Mich., USA. Also preferred is Silicone SM 253 available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetically acceptable compositions of this invention may include volatile hydrocarbon oils. The volatile hydrocarbon comprises from about C.sub.6 to C.sub.22 atoms. A preferred volatile hydrocarbon is an aliphatic hydrocarbon having a chain length from about C.sub.6 to C.sub.16 carbon atoms. An example of such compound includes isohexadecane, under the tradename Permethyl 101 A, available from Presperse, South Plainfield, N.J., USA. Another example of a preferred volatile hydrocarbon is C.sub.12 to C.sub.14 isoparaffin, under the tradename Isopar M, available from Exxon, Baytown, Tex., USA. When used, the volatile hydrocarbons are preferably included in the formulations of this invention at a concentration of 0.1 to 30 weight percent, more preferably 1 to 20 weight percent.

The cosmetically acceptable compositions of this invention may include cationic and ampholytic conditioning polymers. Examples of such include, but are not limited to those listed by the International Cosmetic Ingredient Dictionary published by the Cosmetic, Toiletry, and Fragrance Association (CTFA), 1101 17 Street, N.W., Suite 300, Washington, D.C. 20036. General examples include quaternary derivatives of cellulose ethers, quaternary derivatives of guar, homopolymers and copolymers of DADMAC, homopolymers and copolymers of MAPTAC and quaternary derivatives of starches. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the cosmetically acceptable composition of this invention at a concentration of from 0.1 to 10 weight percent, preferably from 0.2 to 6 weight percent and most preferably from 0.2 to 5 weight percent.

The cosmetically acceptable composition of this invention may include one or more rheological modifiers. The rheological modifiers which can be used in this invention include, but are not limited to high molecular weight crosslinked homopolymers of acrylic acid, and Acrylates/C10-30 Alkyl Acrylate Crosspolymer, such as the Carbopol. and Pemulen series, both available from B. F. Goodrich, Akron, Ohio, USA; anionic acrylate polymers such as Salcare and cationic acrylate polymers such as Salcare SC96, available from Ciba Specialties, High Point, N.C., USA; Acrylamidopropylttrimonium chloride/acrylamide; Hydroxyethyl methacrylates polymers, Steareth-10 Allyl Ether/Acrylate Copolymer; Acrylates/Beheneth-25 Metacrylate Copolymer, known as Aculyn, available from International Specialties, Wayne, N.J., USA; Glyceryl Polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof. When used, the rheology modifiers are preferably included in the cosmetically acceptable composition of this invention at a concentration of from 0.01 to 12 weight percent, preferably from 0.05 to 10 weight percent and most preferably from 0.1 to 6 weight percent.

The cosmetically acceptable composition of this invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The anti oxidants will be present at from 0.01 to 5 weight percent, preferably 0.1 to 3 weight percent and most preferably from 0.2 to 2 weight percent of the cosmetically acceptable composition.

The cosmetically acceptable composition of this invention may include one or more sunscreen active agents. Examples of sunscreen active agents include, but are not limited to octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl PABA, diethanolamine p-methoxy cinnamate, ethyl 4-bis (hydroxypropyl) aminobenzoate, 2-ethylhexy 1-2-cyano-3,3-diphenylacrylate, homomenthyl salicylate, glyceryl aminobenzoate, dihydroxyacetone, octyl dimethyl PABA, 2-phenylbenzimidazole-5-sulfonic acid, triethanolamine salicylate, zinc oxide, and titanium oxide, and mixtures thereof. The amount of sunscreen used in the cosmetically acceptable composition of this invention will vary depending on the specific UV absorption wavelength(s) of the specific sunscreen active(s) used and can be from 0.1 to 10 percent by weight, from 2 to 8 percent by weight.

The cosmetically acceptable composition of this invention may include one or more preservatives. Example of preservatives, which may be used include, but are not limited to 1,2-dibromo-2,4-dicyano butane (Methyldibromo Glutaronitrile, known as MERGUARD. Nalco Chemical Company, Naperville, Ill., USA), benzyl alcohol, imidazolidinyl urea, 1,3-bis (hydroxymethyl)-5,5-dimethyl-2,3-imidazolidinedione (e.g., DMDM Hydantoin, known as GLYDANT, Lonza, Fairlawn, N.J., USA.), methylchloroisothiazolinone and methylisothiazolinone (e.g., Kathon, Rohm & Haas Co., Philadelphia, Pa., USA), methyl paraben, propyl paraben, phenoxyethanol, and sodium benzoate, and mixtures thereof.

The cosmetically acceptable composition of this invention may include any other ingredient by normally used in cosmetics. Examples of such ingredients include, but are not limited to buffering agents, fragrance ingredients, chelating agents, color additives or dyestuffs which can serve to color the composition itself or keratin, sequestering agents, softeners, foam synergistic agents, foam stabilizers, sun filters and peptizing agents.

The surface of pigments, such titanium dioxide, zinc oxide, talc, calcium carbonate or kaolin, can be treated with the unsaturated quaternary ammonium compounds described herein and then used in the cosmetically acceptable composition of this invention. The treated pigments are then more effective as sunscreen actives and for use in color cosmetics such as make up and mascara.

The cosmetically acceptable composition of this invention can be presented in various forms. Examples of such forms include, but are not limited a solution, liquid, cream, emulsion, dispersion, gel, thickening lotion.

The cosmetically acceptable composition of this invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerine, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture. These solvents can be present in proportions of up to as much as 70 percent by weight, for example from 0.1 to 70 percent by weight, relative to the weight of the total composition.

The cosmetically acceptable composition of this invention can also be packaged as an aerosol, in which case it can be applied either in the form of an aerosol spray or in the form of an aerosol foam. As the propellant gas for these aerosols, it is possible to use, in particular, dimethyl ether, carbon dioxide, nitrogen, nitrous oxide, air and volatile hydrocarbons, such as butane, isobutane, and propane.

The cosmetically acceptable composition of this invention also can contain electrolytes, such as aluminum chlorohydrate, alkali metal salts, e.g., sodium, potassium or lithium salts, these salts preferably being halides, such as the chloride or bromide, and the sulfate, or salts with organic acids, such as the acetates or lactates, and also alkaline earth metal salts, preferably the carbonates, silicates, nitrates, acetates, gluconates, pantothenates and lactates of calcium, magnesium and strontium.

Compositions for treating skin include leave-on or rinse-off skin care products such as lotions, hand/body creams, shaving gels or shaving creams, body washes, sunscreens, liquid soaps, deodorants, antiperspirants, suntan lotions, after sun gels, bubble baths, hand or mechanical dishwashing compositions, and the like. In addition to the polymer, skin care compositions may include components conventionally used in skin care formulations. Such components include for example; (a) humectants, (b) petrolatum or mineral oil, (c) fatty alcohols, (d) fatty ester emollients, (e) silicone oils or fluids, and (f) preservatives. These components must in general be safe for application to the human skin and must be compatible with the other components of the formulation. Selection of these components is generally within the skill of the art. The skin care compositions may also contain other conventional additives employed in cosmetic skin care formulations. Such additives include aesthetic enhancers, fragrance oils, dyes and medicaments such as menthol and the like.

The skin care compositions of this invention may be prepared as oil-in-water, water-in-oil emulsions, triple emulsions, or dispersions.

Preferred oil-in-water emulsions are prepared by first forming an aqueous mixture of the water-soluble components, e.g. unsaturated quaternary ammonium compounds, humectants, water-soluble preservatives, followed by adding water-insoluble components. The water-insoluble components include the emulsifier, water-insoluble preservatives, petrolatum or mineral oil component, fatty alcohol component, fatty ester emollient, and silicone oil component. The input of mixing energy will be high and will be maintained for a time sufficient to form a water-in-oil emulsion having a smooth appearance (indicating the presence of relatively small micelles in the emulsion). Preferred dispersions are generally prepared by forming an aqueous mixture of the water-soluble components, followed by addition of thickener with suspension power for water-insoluble materials.

Compositions for treating hair include bath preparations such as bubble baths, soaps, and oils, shampoos, conditioners, hair bleaches, hair coloring preparations, temporary and permanent hair colors, color conditioners, hair lighteners, coloring and non-coloring hair rinses, hair tints, hair wave sets, permanent waves, curling, hair straighteners, hair grooming aids, hair tonics, hair dressings and oxidative products. The dispersion polymers may also be utilized in styling type leave-in products such as gels, mousses, spritzes, styling creams, styling waxes, pomades, balms, and the like, either alone or in combination with other polymers or structuring agents in order to provide control and hair manageability with a clean, natural, non-sticky feel.

Hair care compositions of this invention give slippery feel and that can be easily rinsed from the hair due to the presence of the dispersion polymer, volatile silicones, other polymers, surfactants or other compounds that may alter the deposition of materials upon the hair.

In the case of cleansing formulations such as a shampoo for washing the hair, or a liquid hand soap, or shower gel for washing the skin, the compositions contain anionic, cationic, nonionic, zwitterionic or amphoteric surface-active agents typically in an amount from about 3 to about 50 percent by weight, preferably from about 3 to about 20 percent, and their pH is general in the range from about 3 to about 10.

Preferred shampoos of this invention contain combinations of anionic surfactants with zwitterionic surfactants and/or amphoteric surfactants. Especially preferred shampoos contain from about 0 to about 16 percent active of alkyl sulfates, from 0 to about 50 weight percent of ethoxylated alkyl sulfates, and from 0 to about 50 weight percent of optional surface-active agents selected from the nonionic, amphoteric, and zwitterionic surface-active agents, with at least 5 weight percent of either alkyl sulfate, ethoxylated alkyl sulfate, or a mixture thereof, and a total surfactant level of from about 10 weight to about 25 percent.

The shampoo for washing hair also can contain other conditioning additives such as silicones and conditioning polymers typically used in shampoos. U.S. Pat. No. 5,573,709 provides a list of non-volatile silicone conditioning agents that can be used in shampoos. The conditioning polymers for use with the present invention are listed in the Cosmetic, Toiletries and Fragrance Associations (CTFA) dictionary. Specific examples include the Polyquaterniums (example Polyquaternium-1 to Polyquaternium-50), Guar Hydroxypropyl Trimonium Chloride, Starch Hydroxypropyl Trimonium Chloride and Polymethacrylamidopropyl Trimonium Chloride.

Other preferred embodiments consist of use in the form of a rinsing lotion to be applied mainly before or after shampooing. These lotions typically are aqueous or aqueous-alcoholic solutions, emulsions, thickened lotions or gels. If the compositions are presented in the form of an emulsion, they can be nonionic, anionic or cationic. The nonionic emulsions consist mainly of a mixture of oil and/or a fatty alcohol with a polyoxyethyleneated alcohol, such as polyoxyethyleneated stearyl or cetyl/stearyl alcohol, and cationic surface-active agents can be added to these compositions. The anionic emulsions are formed essentially from soap.

If the compositions are presented in the form of a thickened lotion or a gel, they contain thickeners in the presence or absence of a solvent. The thickeners which can be used are especially resins, Carbopol-type acrylic acid thickeners available from B.F. Goodrich; xanthan gums; sodium alginates; gum arabic; cellulose derivatives and poly-(ethylene oxide) based thickeners, and it is also possible to achieve thickening by means of a mixture of polyethylene glycol stearate or distearate or by means of a mixture of a phosphoric acid ester and an amide. The concentration of thickener is generally 0.05 to 15 percent by weight. If the compositions are presented in the form of a styling lotion, shaping lotion, or setting lotion, they generally comprise, in aqueous, alcoholic or aqueous-alcoholic solution, the ampholyte polymers defined above.

In the case of hair fixatives, the composition may also contain one or more additional hair fixative polymers. When present, the additional hair fixative polymers are present in a total amount of from about 0.25 to about 10 percent by weight. The additional hair fixative resin can be selected from the following group as long as it is compatible with a given dispersion polymer: acrylamide copolymer, acrylamide/sodium acrylate copolymer, acrylate/ammonium methacrylate copolymer, an acrylate copolymer, an acrylic/acrylate copolymer, adipic acid/dimethylaminohydroxypropyl diethylenetriamine copolymer, adipic acid/epoxypropyl diethylenetriamine copolymer, allyl stearate/VA copolymer, aminoethylacrylate phosphate/acrylate copolymer, an ammonium acrylate copolymer, an ammonium vinyl acetate/acrylate copolymer, an AMP acrylate/diacetoneacrylamide copolymer, an AMPD acrylate/diacetoneacrylamide copolymer, butyl ester of ethylene/maleic anhydride copolymer, butyl ester of PVM/MA copolymer, calcium/sodium PVM/MA copolymer, corn starch/acrylamide/sodium acrylate copolymer, diethylene glycolamine/epichlorohydrin/piperazine-copolymer, dodecanedioic acid/cetearyl alcohol/glycol copolymer, ethyl ester of PVM/MA copolymer, isopropyl ester of PVM/MA copolymer, karaya gum, a methacryloyl ethyl betaine/methacrylate copolymer, an octylacrylamide/acrylate/butylaminoethyl methacrylate copolymer, an octylacrylamide/acrylate copolymer, phthalic anhydride/glycerin/glycidyl decanoate copolymer, a phthalic/trimellitic/glycol copolymer, polyacrylamide, polyacrylamidomethylpropane sulfonic acid, polybutylene terephthalate, polyethylacrylate, polyethylene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-39, polyquaternium-47, polyvinyl acetate, polyvinyl butyral, polyvinyl imidazolinium acetate, polyvinyl methyl ether, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, shellac, sodium acrylates copolymer, sodium acrylates/Acryinitrogens copolymer, sodium acrylate/vinyl alcohol copolymer, sodium carrageenan, starch diethylaminoethyl ether, stearylvinyl ether/maleic anhydride copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, a vinyl acetate/crotonate copolymer, vinyl acetate/crotonic acid copolymer, vinyl acetate/crotonic acid/methacryloxybenzophenone-1 copolymer, vinyl acetate/crotonic acid/vinyl neodecanoate copolymer, and mixtures thereof. Synthetic polymers used for creating styling aids are described in “The History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

The cosmetic compositions of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonites, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane, butane, diethyl ether, or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension may comprise liquid diluents, for example water, ethanol or propylene glycol, suspending agents, for example ethoxylated isosteary alcohols, polyoxyethylene sorbitol esters and poly oxyethylene sorbitan esters, micocrystalline cellulose, aluminum metahydroxide, bentonite, agar and tragacanth or mixtures of these ingredients.

The formulation of cleansing compositions with surfactant may comprise aliphatic alcohol sulfate, aliphatic alcohol ether sulfate, sulfosucinnate monoester, isothinate, imidazolium derivatives, methyltaurate, sarcocinate, fatty acid amide ether sulfate, alkyl amido betain, aliphatic alcohol, fatty acid glyceride, fatty acid diethanolamide, vegetable oil, lanoline derivatives, ethoxylated glycerol fatty acid ester or mixtures of these ingredients.

Additional antioxidant ingredients and compositions can be selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The blood micro-circulation improvement ingredients and compositions can be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The anti-inflammatory ingredients or compositions can be selected from, but not limited to, at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

Certain divalent and polyvalent metal ions can also be present in the compositions of the present invention. The examples of such metal ions include zinc, copper, manganese, vanadium, chromium, cobalt, and iron.

The amount of MMP inhibitor in the compositions can be from 0.01% to 100% of composition. This is because MMP inhibitor can be used as is in the form of a formulation, for example, as a 100% MMP inhibitor “Super Strength” wound healing composition or body deodorizing powder, or 0.01% in a sore and cracks healing lip balm.

The efficacy of MMP inhibitors of the present invention has been determined by a new procedure discovered by the present inventor. It is based on the inhibition of a tyrosinase enzyme based model system, for example Canters et al., “Kinetic and paramagnetic NMR investigations of the inhibition of Streptomyces antibioticus tyosinase”, Journal of Molecular Catalysis, B: Enzymatics, vol. 8, 27-35 (2000). Tyrosinase is a copper-based monooxygenase enzyme that catalyzes the hydroxylation of monophenols (hydroxybenzenes) and the oxidation of ortho-diphenols to ortho-quinones. The active-site of tyrosinase is known to contain two copper ions (CuA and CuB). Each of the two copper ions has been shown to be bound by three conserved histidine residues. The regions around these copper-binding ligands are well conserved. Moreover, the distance between these two copper ions is 26 Angstrom units [(van Amsterdam et al., Angewandte Chemie, 42: 62-64 (2003); Bubacco et al., J. Biol. Chem., 181-194 (2003)]. At least two proteins related to tyrosinase are known to exist in mammals, and include TRP-1, which is responsible for the conversion of 5,6-dihydroxyindole-2-carboxylic acid (DHICA) to indole-5,6-quinone-2-carboxylic acid (IQCA) or indole-5,6-quinone (IQ); and TRP-2, which is the melanogenic enzyme DOPAchrome tautomerase that catalyzes the conversion of DOPAchrome to DHICA. TRP-2 differs from tyrosinases and TRP-1 in that it binds two zinc ions instead of copper. Other proteins that belong to this family are plant polyphenol oxidases (PPO), which catalyze the oxidation of mono- and ortho-diphenols to ortho-diquinone. Thus, by using TRP-2 as a model, the present inventor has found that MMP inhibitors can be tested by evaluation of the inhibition of color formation from the action of TRP-2 on tyrosine. The colorless tyrosine is converted into yellow/orange colored ortho-diquinone of tyrosine, which is spectrophotometrically quantitated and its rate kinetics thus established. Since TRP-2 is based on two zinc atoms, which is very similar to MMP enzymes that also contain two zinc atoms albeit in a possibly different spatial geometry, this rate kinetics obtained from TRP-2 inhibition of MMP inhibitors of the present invention can also be extended as a predictive methodology to MMP inhibition. This constitutes a new procure for the rapid screening and discovery of new MMP inhibitors. For example, for a wound healing application for MMP-2 and MMP-9 inhibition, the following order of efficacy was noted for some of the compounds disclosed in claims section: 2,6-Dihydroxyacetophenone>2,5-Dihydroxyacetophenone>2,4-Dihydroxyacetophenone>2-Hydroxy-4-methoxyaceophenone >>>Arbutin. The oxime derivatives of these acetophenones had a very similar order of efficacy: 2,6-Dihydroxyacetophenone Oxime>2,5-Dihydroxyacetophenone Oxime>2,4-Dihydroxyacetophenone Oxime>2-Hydroxy-4-methoxyaceophenone Oxime. However, the oxime derivatives of these acetophenones in general were more efficacious than their corresponding acetophenones themselves.

It is not totally clear at this time if the MMP inhibitors of the present invention are causing the blocking of both copper and zinc actives-sites in tyrosinase model described herein. It is also not clear if the MMP inhibitors of the present invention are acting as a competitive substrates, or causing changes in the environment of two zinc atoms, or two copper atoms, or both, of tyrosinase enzyme model, since that can also cause a disruption in the enzymatic activity of tyrosinase model. Irrespective of the precise mechanisms involved, the value and validity of present tyrosinase test model to evaluate and discover new MMP inhibitors is both unprecedented and surprising.

### EXAMPLES

The following examples are presented to illustrate presently preferred practice thereof. These examples also include the formulation of consumer desirable lotion, cream, and other such compositions for their retail marketing. As illustrations they are not intended to limit the scope of the invention. All quantities are in weight %.

### Example 1

### MMP Inhibiting Hair Growth Retardant Serum

Ingredients % Weight (1) Deionized water 20.0 (2) 2-Acetyl-8-hydroxyquinoxaline 5.0 (3) Methylpropanediol 69.5 (4) Dimethicone copolyol 4.0 (5) Preservatives 0.5 (6) Ammonium Acryloyidimethyltaurate/vp copolymer 1.0. Procedure. Make main batch by mixing (2) to (5) at room temperature. Pre-mix (1) and (6) to a clear paste and add to main batch with mixing. The product has a clear to slightly hazy syrup-like appearance, typical of a skin serum product. It is absorbed rapidly with a silky smooth skin feel.

### Example 2

### Wound Healing Serum with Copper Ions

Ingredients % Weight (1) Deionized water 20.0 (2) Quinacetophenone 5.0 (3) Methylpropanediol 69.0 (4) Dimethicone copolyol 4.0 (5) Preservatives 0.5 (6) Copper Gluconate 0.5. (7) Ammonium Acryloyidimethyltaurate/VP copolymer 1.0 Procedure. Make main batch by mixing (2) to (6) at room temperature. Pre-mix (1) and (7) to a clear paste and add to main batch with mixing. The product has a clear to slightly hazy syrup-like light blue appearance, typical of a skin serum product. It is absorbed rapidly with a silky smooth skin feel.

### Example 3

### Wound Healing Cream

Ingredients % Weight (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Caprylic/capric triglyceride 5.0 (6) Resacetopheenone 3.0 (7) Paeonol 1.0 (8) (8) Preservatives 0.5. Procedure. Mix 1 to 5 and heat to 75-80° C. Adjust pH to 4.0 4.5. Cool to 35-40 C with mixing. Add 6 to 8 with mixing. Adjust pH to 4.0-4.5, if necessary. White to off-white cream.

### Example 4

### Collagen Boosting Antiaging Facial Mask Composition

Ingredient. % (1) Chitosan 5.0 (2) 2,5-Dihydroxy acetophenone Oxime 5.0 (3) Glycerin 17.7 (4) Water 70.6 (5) Yohimbine HCl 0.5 (6) Niacinamide Lipoate 0.5 (7) Glutathione0.2 (8) Preservatives 0.5 Procedure: Mix 1, 2, and 3 to a paste. Mix 4 to 8 separately to a clear solution. Add this to main batch and mix. A clear gel product is obtained. It is applied on the face and neck and left for 10 to 30 minutes, then rinsed off.

### Example 5

### Skin Discoloration and Age Spots Cure Cream

Ingredient % (1) Water 65.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Niacinamide Lactate 1.0 (16) Niacinamide Hydroxycitrate 3.1 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Polysorbate-20 2.0 (23) Sepigel-305 2.0. Procedure. Mix (1) to (13) and heat at 70 to 80 C till homogenous. Cool to 40 to 50 C. Premix (14) to (16) and add to batch with mixing. Add all other ingredients and mix. Cool to room temperature. An off-white cream is obtained.

### Example 6

### Anti-inflammatory Acne Cream

Ingredient % (1) Water 62.3 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Niacinamide Salicylate 4.0 (16) Niacinamide Hydroxycitrate 2.2 (17) 2,4-Dihydroxy Acetophenone (Resacetophenone) 1.1 (18) Paeonol 1.5 (19) Carnosine 0.1 (20) Cyclomethicone, Dimethicone Crosspolymer 2.0 (21) Arbutin 0.5 (22) Pyridoxine Salicylate (23) Polysorbate-20 2.0 (24) Sepigel-305 2.0. Procedure. Mix (1) to (13) and heat at 70 to 80 C till homogenous. Cool to 40 to 50 C. Premix (14) to (16) and add to batch with mixing. Add all other ingredients and mix. Cool to room temperature. An off-white cream is obtained.

### Example 7

### Anti-inflammatory Skin Brightening Cleanser

Ingredient % (1) PEG-6 63.329 (2) Hydroxypropyl Cellulose 0.3 (3) Boswellia Serrata 0.05 (4) Sodium Cocoyl Isethionate 20.0 (5) Sodium Lauryl Sulfoacetate 5.0 (6) L-Glutathione 0.01 (7) Resveratrol 0.01 (8) 2,5-DihydroxyAcetophenone 0.1 (9) 2,6-Dihydroxy Acetophenone 0.001 (10) Ascorbic acid 10.0 (11) Phenoxyethanol 0.7 (12) Ethylhexylglycerin 0.3 (13)Fragrance 0.2. Procedure. Mix (1) and (2) to a clear thin gel. Add all other ingredients and mix in a homogenizer. A white cream-like cleanser is obtained.

### Example 8

### Arthritis Pain Relief Anti-inflammatory Gel

Ingredients % (1) C12-15 Alkyl Benzoate 67.75 (2) Ethylenediamine/Hydrogenated Dimer Dilinoleate Copolymer Bis-Di-C14-18 Alkyl Amide 10.0 (3) Ximenia Oil 0.1 (4) Capsaicin 0.25 (5) Magnolol (and Honokiol 0.2 (6) Paeonol 0.5 (7) Tetrahydrocurcuminoids 0.2 (8) Zeolite 20.0 (9) Fragrance 1.0. Procedure. Mix (1) and (2) and heat at 80 to 90 C till clear. Cool to 40 to 50 C and add all other ingredients and mix. Cool to room temperature. A white gel-like product is obtained.

### Example 9

### Arthritis Anti-inflammatory Transparent Gel

Ingredients % (1) C12-15 Alkyl Benzoate 96.75 (2) Dibutyl Lauroyl Glutamide 1.0 (3) Ximenia Oil 0.1 (4) Capsaicin 0.25 (5) Magnolol (and Honokiol 0.2 (6) Paeonol 0.5 (7) Tetrahydrocurcuminoids 0.2 (8) Fragrance 1.0. Procedure. Mix (1) and (2) and heat at 95 to 110 C till clear. Cool to 40 to 50 C and add all other ingredients and mix. Cool to room temperature. A transparent gel-like product is obtained.

### Example 10

### Topical Anesthetic Spray Lotion with Anti-inflammatory Agents

Ingredients % (1) PEG-4 81.0 (2) Benzocaine 16.0 (3) Fragrance 0.5 (4) Paeonol 0.5 (5) 2,4-Dihydroxy Acetophenone 2.0. Procedure. Mix all ingredients ill a clear solution is obtained. Fill in spray bottles.

### Example 11

### Anti-inflammatory Color-Changing Acne Mask with Controlled Release

Ingredients % (1) Grapeseed oil 34.28 (2) Ethylenediamine/Hydrogenated Dimer Dilinoleate Copolymer Bis-Di-C14-18 Alkyl Amide 5.0 (3) Dimthicone 2.0 (4) Propyl Paraben 0.3 (5)Jojoba oil 0.5 (6) Sweet Almond oil 4.0 (7) Shea butter 0.2 (8) Mango butter 0.2 (9) Avocado utter 0.2 (10) Murumuru butter 0.2 (11) Color Change Green/Blue dye 0.01 (12) Niacinamide Hydroxybenzoate 5.5 (13) Vitamin E 0.11 (14) Phenoxyethanol 0.7 (15) Zeolite 31.0 (16) Ethylhexylglycerin 0.5 (17) Laureth-3 15.0 (18) Fragrance 0.5. Procedure. Mix (1) to (10) and heat at 70 to 80 C till clear. Cool to 35 to 45 C and all other ingredients and mix. Cool to room temperature. A light green thin paste is obtained. Upon contact with water, it turns blue and releases heat.

### Example 12

### Hair Growth Promoting Shampoo

Ingredient % (1) Water 64.2 (2) 2-Acetylpyridine N-oxide (1.2) (3) Sodium Lauryl Sulfoacetatel 0.0 (4) Disodium Laureth Sulfosuccinate 20.0 (5) Phenoxyethanol 0.7 (6) Chlorphenesin 0.3 (7) PEG-120 Methyl Glucose Dioleate 2.5. (8) Hydrolyzed Soy Protein 0.5 (9) Hydrolyzed Silk Protein 0.5 (10) Oat Extract 0.1. Procedure. Mix (1) to (7) and heat at 60 to 70 C to a clear solution. Cool to 35 to 40 C and add all other ingredients and mix. Cool to room temperature.

### Example 13

### Topical Inflammation Control Massage Lotion

Ingredients % (1) Water 39.158 (2) Acrylates/C10-30 Alkyl Acrylate Crosspolymer 0.5 (3) Escin 0.1 (4) Sodium Stearyl Phthalamate 1.0 (5) Sodium Hydroxide 0.142 (6) Cetyl Alcohol 4.0 (7) Phenoxyethanol 0.7 (8) Chlorphenesin 0.3 (9) Grapeseed oil 10.0 (10) Ethylhexylglycein 0.5 (11) Polysorbate-20 10.0 (12) PEG-6 2.0 (13) Tetrahydrocurcuminoids 0.1 (14) Magnolol 0.1 (15) Paeonol 0.2 (16) Fragrance 1.0. Procedure. Mix (1) to (11) and heat at 80 to 90 C till clear. Cool to 45 to 55. Pre-mix (12) to (16) and add to main batch and mix. Cool to room temperature and adjust pH to 7.5.

### Example 14

### Anti-Inflammatory Make-Up Remover Fluid

Ingredients % (1) Water 39.158 (2) Acrylates/C10-30 Alkyl Acrylate Crosspolymer 0.5 (3) Harpagoside 0.1 (4) Sodium Stearyl Phthalamate 1.0 (5) Sodium Hydroxide 0.142 (6) Cetyl Alcohol 4.0 (7) Phenoxyethanol 0.7 (8) 1,2-Octanediol 0.3 (9) Grapeseed oil 10.0 (10) Methyl Soyate 30.0 (11) Ethylhexylglycein 0.5 (12) Polysorbate-20 10.0 (13) PEG-6 2.0 (14) Tetrahydrocurcuminoids 0.1 (15) Magnolol 0.1 (16) Paeonol 0.2 (17) Fragrance 1.0. Procedure. Mix (1) to (12) and heat at 80 to 90 C till clear. Cool to 45 to 55. Pre-mix (13) to (16) and add to main batch and mix. Add (17) and mix. Cool to room temperature and adjust pH to 7.5.

### BRIEF DESCRIPTION OF THE DRAWINGS

[FIG. 1] MMP inhibitors claimed by Chen et al.

[FIG. 2] MMP inhibitors claimed by Stallings et al.

[FIG. 3] MMP inhibitors claimed by Curtin et al.

[FIG. 4] MMP inhibitors claimed by Dublanchet et al.

[FIG. 5] MMP inhibitors claimed by O'Brien et al.

[FIG. 6] MMP inhibitors claimed by Newton et al.

[FIG. 7] MMP inhibitors claimed by Baarlam et al.

[FIG. 8] MMP inhibitors claimed by Barlaam et al.

[FIG. 9] MMP inhibitors claimed by Becker et al.

[FIG. 10] MMP inhibitors claimed by Klingler et al.

[FIG. 11] MMP inhibitors claimed by VanZandt et al.

[FIG. 12] MMP inhibitors claimed by Bunker et al.

[FIG. 13] MMP inhibitors claimed by Bunker et al.

[FIG. 14] MMP inhibitors claimed by Ott et al.

[FIG. 15] MMP inhibitors claimed by King et al.

[FIG. 16] MMP inhibitors claimed by Hayakawa et al.

[FIG. 17] MMP inhibitors claimed by Johnson et al.

[FIG. 18] MMP inhibitors claimed by Heinicke et al.

[FIG. 19] MMP inhibitors claimed by Gaudilliere et al.

[FIG. 20] MMP inhibitors claimed by Arnold et al.

[FIG. 21] Mechanism of protease action.

[FIG. 22] Mechanism of zinc MMP catalyzed peptide bond cleavage.

[FIG. 23] Hydroxyaryl Alkyl Ketones and their derivatives.

[FIG. 24] Nitrogen Hetero-aromatic Alkyl Ketones and their derivatives.

[FIG. 25] N-Hetero-aromatic substituted alkyl ketones.

[FIG. 26] Additional N-hetero-aromatic substituted alkyl ketones.

[FIG. 27] Additional N-hetero-aromatic substituted alkyl ketones.

[FIG. 28] Additional N-hetero-aromatic substituted alkyl ketones.

[FIG. 29] Spatial distortion of Zinc active-site of MMP by a hydroxyaryl alkyl ketone.

[FIG. 30] Stages of wound healing process.

What is claimed is:

1. A topical Matrix Metalloprotease Inhibitor (MMP) composition comprising; (i) At least one hydroxyaryl or polyhydroxyaryl compound that contains an alkyl carbon side chain with a ketone group attached at the first carbon atom of the alkyl side chain, and said ketone group is directly attached to the aromatic ring at a position adjacent to at least one hydroxyl group of hydroxyaryl or polyhydroxyaryl ring; or at least one N-hetero-aromatic compound that contains an alkyl carbon side chain with a ketone group attached at the first carbon atom of the alkyl side chain, and said ketone group is directly attached to the nitrogen hetero-aromatic ring at a position adjacent to the aromatic ring nitrogen atom; or a combination thereof; and (ii) A cosmetically or pharmaceutically acceptable topical delivery system or carrier base composition.

2. A composition according to claim 1, wherein hydroxyaryl compound is selected from hydroxy or polyhydroxy acetophenones, or hydroxy or Polyhydroxy propiophenones, and their substituted derivatives.

3. A composition according to claim 1, wherein-hetero-aromatic compound is selected from 2-acetyl-substituted N-hetero-aromatic compounds.

4. A composition according to claim 1, wherein hydroxyaryl compound is selected from oxime, or hydrazide, or semicarbazone, or oxamic hydrazone derivatives of hydroxyaryl- or polyhydroxyaryl alkyl ketones.

5. A composition according to claim 1, wherein N-hetero-aromatic compound is selected from oxime, or hydrazide, or semicarbazone, or oxamic hydrazone derivatives of N-hetero-aromatic alkyl ketones.

6. A composition according to claim 1, wherein hydroxyaryl compound contains additional cyclic rings attached at the aromatic ring.

7. A composition according to claim 1, wherein N-hetero-aromatic compound contains additional cyclic rings attached at the nitrogen hetero-aromatic ring.

8. A composition according to claim 1, wherein N-hetero-aromatic compound contains additional hetero-atoms in same ring that contains nitrogen hetero-atom; or in other cyclic ring or rings that are attached to the nitrogen hetero-aromatic ring.

9. A composition according to claim 1, wherein the cosmetically or pharmaceutically acceptable delivery system can be traditional water and oil emulsions, suspensions, colloids, microemulsions, clear solutions, suspensions of nanoparticles, emulsions of nanoparticles, powders, or anhydrous compositions.

10. A composition according to claim 2, wherein hydroxy or polyhydroxy acetophenone compound is selected from 2-hydroxyacetophenone, 3-hydroxyacetophenone, 4-hydroxyacetophenone, 2,3-dihydroxyacetophenone, 2,4-dihydroxyacetophenone, 2,5-dihydroxyacetophenone, 2,6-dihydroxyacetophenone, 3,4-dihydroxyacetophenone, 3,5-dihydroxyacetophenone, 2,4,6-trihydroxyacetophenone, 2,3,4-trihydroxyacetophenone, 2,3,5-trihydroxyacetophenone, 2,3,6-trihydroxyacetophenone, 2,4,5-trihydroxyacetophenone, 3,4,5-trihydroxyacetophenone, Resacetophenone, 2-Acetyl resorcinol, 4-Acetyl resorcinol, 3,4-Dihydroxyacetophenone, acetyl quinol, Quinacetophenone, 1-(3-Hydroxy-4-methoxy-5-methylphenyl) ethanone, 1-(3-hydroxy-4-methoxyphenyl) ethanone, Paeonol, 5′-Bromo-2′-hydroxyacetophenone, 5′-Chloro-2′-hydroxyacetophenone, 3′,5′-Dichloro-2′-hydroxyacetophenone, 3′,5′-Dibromo-4′-hydroxyacetophenone, 5-Chloro-3-bromo-2-hydroxyacetophenone, and combinations thereof.

11. A composition according to claim 2, wherein hydroxy or polyhydroxy propiophenone compound is selected from 2-hydroxypropiophenone, 3-hydroxypropiophenone, 4-hydroxypropiophenone, 2,3-dihydroxypropiophenone, 2,4-dihydroxypropiophenone, 2,5-dihydroxypropiophenone, 2,6-dihydroxypropiophenone, 3,4-dihydroxypropiophenone, 3,5-dihydroxypropiophenone, 2,4,6-trihydroxypropiophenone, 2,3,4-trihydroxypropiophenone, 2,3,5trihydroxypropiophenone, 2,3,6-trihydroxypropiophenone, 2,4,5-trihydroxypropiophenone, 3,4,5-trihydroxypropiophenone, 1-(2,4-dihydroxyphenyl)-2-hydroxyethanone, (2-hydroxyphenyl)(oxo)acetic acid, 1-(2,6-dihydroxyphenyl)-1-butanone, 1-(1-hydroxy-2-naphthyl)ethanone, 1-(2-hydroxy-1-naphthyl)ethanone, 5,7-dihydroxy-1-indanone, 1-(2-hydroxy-5-methylphenyl)-1,3-butanedione, N-(4-acetyl-3-hydroxyphenyl)acetamide, 4-acetyl-3-hydroxyphenyl acetate, 1,1′-(4,6-Dihydroxy-1,3-phenylene)bisethanone, 1-(1-hydroxy-2-naphthyl)ethanone, 2,3-Dihydro-9,10-dihydroxy-1,4-anthracenedione, and combinations thereof.

12. A composition according to claim 2, wherein oxime, or oxime O-alkyl ether, or hydrazone, or semicarbazone, or oxamic hydrazone derivatives of hydroxy or polyhydroxy acetophenones, or hydroxy or polyhydroxy propiophenones, or combinations thereof, are selected.

13. A composition according to claim 2, wherein oxime, or oxime O-alkyl ether, or hydrazone, or semicarbazone, or oxamic hydrazone derivatives of 2-acetyl-N-heteroaromatic, or 2-propionyl-N-heteroaromatic compounds, or combinations thereof, are selected.

14. A composition according to claim 3, wherein N-hetero-aromatic compound is selected from 2-acetylpyridine, 2-Acetyl-4-methylpyridine, 1-(1-oxido-2-pyridinyl)ethanone, 2,6-Diacetylpyridine, 3-(Dimethylamino)-1-(2-pyridyl)-2-propen-1-one, 1,8-Diazafluoren-9-one, 2-phenyl-1-(2-pyridinyl) ethanone, 3-phenyl-1-(2-pyridinyl)-2-propen-1-one, 1-(2-pyridinyl)-3-(3-pyridinyl)-2-propen-1-one, 2-hydroxy-1,2-di(2-pyridinyl)ethanone, 1-(2-pyridinyl)-3-(2-thienyl)-2-propen-1-one, 3-(2-hydroxy phenyl)-(2-pyridinyl)-2-propen-1-one, 3-(1-oxido-2-pyridinyl)-1-(2-pyridinyl)-2-propen-1-one, 2-acetylpyrrole, 2-Acetyl-1-methylpyrrole, 2-chloro-1-(1H-pyrrol-2-yl)ethanone, 2-(Trifluoroacetyl)pyrrole, 1,4-dihydrocyclopenta[b]indol-3(2H)-one, 2,3,4,9-tetrahydro-1H-carbazol-1-one, (2E)-1,3-di(H-pyrrol-2-yl)-2-propen-1-one, (2E)-3-phenyl-1-(1H-pyrrol-2-yl)-2-propen-1-one, 2-acetylimidazole, 1-(5-methyl-2-phenyl-1H-imidazol-4-yl)ethanone, 1-(5,6-dimethyl-1H-benzimidazol-2-yl)ethanone, (4-chlorophenyl)(1-methyl-1H-imidazol-2-yl)methanone, (2E)-1-(1H-benzimidazol-2-yl)-3-(4-pyridinyl)-2-propen-1-one, 1-[1-(4-methylbenzyl)-1H-benzimidazol-2-yl]ethanone, (2E)-1-(1H-benzimidazol-2-yl)-3-(2-fluorophenyl)-2-propen-1-one, (2E)-1-(1H-benzimidazol-2-yl)-3-(2-chlorophenyl)-2-propen-1-one, (2E)-1-(5-chloro-1H-benzimidazol-2-yl)-3-phenyl-2-propen-1-one, 1-[1-(2-chlorobenzyl)-1H-benzimidazol-2-yl]ethanon, (2E)-1-(5,6-dichloro-1H-benzimidazol-2-yl)-3-(4-pyridinyl)-2-propen-1-one, 2-acetylthiazole, 1-(1,3-benzothiazol-2-yl)ethanone, 2-acetylpyrimidine, 2-acetylindole, 2-acetyl-1-methylpyrrole, 2-acetyl-4-methylpyridine, 1-acetylphenothiazine, 2-hydroxy-1-acetylphenothiazine, 8-hydroxy-9-acetylphenanthrene, 2-acetylpyrazine, 1-(3-methyl-2-pyrazinyl)ethanone, 2-acetylquinoline, 2-acetyl-8-hydroxyquinoline, 2-acetyltryptophane, 2-acetyltryptophanamide, 2-acetylpyridine N-oxide, 2-acetylquinazoline, 2-acetylquinoxaline, 3-acetylpyridazine, 6,6′-diacetyl-2,2′-pyridyl, 3-acetyl-1,2,4-trizol, and combinations thereof.

15. A composition according to claim 4, wherein oxime derivatives of hydroxyaryl compositions are selected from 2-hydroxyacetophenone oxime, 2,3-dihydroxyacetophenone oxime, 2,4-dihydroxyacetophenone oxime, 2,5-dihydroxyacetophenone oxime, Resacetophenone oxime, acetyl quinol oxime, Quinacetophenone oxime, Paeonol oxime, 2-hydroxypropiophenone oxime, 2,3-dihydroxypropiophenone oxime, 2,4-dihydroxypropiophenone oxime, 2,5-dihydroxypropiophenone oxime, 7-acetyl-5,8-dihydroxyquinoline oxime, and combinations thereof.

16. A composition according to claim 6, wherein hydroxyaryl compound is selected from 1-hydroxy-2-acetyinaphthalene; 1-hydroxy-2-acetyl-5,6,7,8-tetrahydro-naphthalene; 7-acetyl-8-hydroxyquinoline; 3-acetyl-4-hydroxyacridine; 6-acetyl-7-hydroxybenzothiazole, and combinations thereof.

17. A compound according to claim 8, wherein N-hetero-aromatic compound contains additional hetero-atoms that are selected from N, S, or O, or combinations thereof, in same ring that contains nitrogen hetero-atom, or in other cyclic ring or rings that are attached to the nitrogen hetero-aromatic ring.

18. A composition according to claim 9, wherein cosmetically or pharmaceutically acceptable topical delivery system or carrier base composition additionally contains hydroxy or polyhydroxy flavones, hydroxy or polyhydroxy coumarins, hydroxy or polyhydroxy isoflavones, hydroxy or polyhydroxy chromanones, and hydroxy or polyhydroxy chromones, and combinations thereof.

19. A composition according to claim 9, wherein a cosmetically or pharmaceutically acceptable topical delivery system or carrier base composition additionally contains a divalent or a polyvalent metal ion or combinations thereof.

20. A composition according to claim 9, wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

21. A composition according to claim 19, wherein divalent metal ions are selected from copper, zinc, iron, selenium, vanadium, manganese, and combinations thereof.

22. A composition according to claim 20, wherein anti-inflammatory agents are selected from Boswellia serrata, Corosolic acid (Banaba), Ursolic acid, Oleanolic acid, Salicinol (Salacia), Rosmarinic acid, Ruscogenins, Darutoside, Asiaticoside, Sericoside, Harpagoside (Devil's Claw), Magnolia Bark (Honokiol, Magnolol), Horse Chestnut (Escin, Esculin), Ginger (Gingerol), Turmeric Extract (Tetrahydrocurcuminoids), Corydalis, Myricetin, and combinations thereof.
